(19) United States
(12) Patent Application Publication
(10) Pub. No.: US 2013/0116445 A1
(43) Pub. Date: May 9, 2013

(54) TRIAZOLIUM CARBENE CATALYSTS AND PROCESSES FOR ASYMMETRIC CARBON-CARBON BOND FORMATION

(71) Applicants: Tomislav Rovis, Fort Collins, CO (US); Daniel Dirocco, Fort Collins, CO (US); Joseph Guiles, Lafayette, CO (US)

(72) Inventors: Tomislav Rovis, Fort Collins, CO (US); Daniel Dirocco, Fort Collins, CO (US); Joseph Guiles, Lafayette, CO (US)

(73) Assignee: Colorado State University Research Foundation, Fort Collins, CO (US)

(21) Appl. No.: 13/809,741

(22) PCT Filed: Jul. 12, 2011
(86) PCT No.: PCT/US11/43735

§ 371 Date: Jan. 17, 2013

Related U.S. Application Data
(60) Provisional application No. 61/363,331, filed on Jul. 12, 2010.
(60) Provisional application No. 61/497,424, filed on Jun. 15, 2011.

(51) Int. Cl. C07D 249/16

(52) U.S. Cl. CPC C07D 249/16
USPC 548/262.4; 568/306; 568/307; 568/704; 564/186

(57) ABSTRACT

Provided herein are chiral triazolium catalysts useful for asymmetric C—C bond formation and processes for their preparation. Also provided are synthetic reactions in which these catalysts are used, in particular, in asymmetric C—C bond formation.

### RELATED APPLICATIONS

This application claims priority to U.S. Provisional Patent Application No. 61/363,331, entitled “Stetter Reaction Using Novel Fluorinated Catalysts”, filed Jul. 12, 2010 and to U.S. Provisional Patent Application No. 61/497,424, entitled “Fluorine Modified Amine Heterocycles and Azolium Catalysts”, filed Jun. 15, 2011, each of which is incorporated by reference in its entirety. This application is related to U.S. patent application Ser. No. 13/020,693, entitled “Triazolium Carbene Catalysts and Stereoselective Bond Forming Reactions Thereof”, filed Feb. 3, 2011, which claims priority to U.S. Provisional Patent Application No. 61/300,905, entitled “N-heterocyclic Carbenc Catalyzed Asymmetric Hydration Direct Synthesis of Select Alpha-Proteo and Alpha-Deutero Carboxylic Acids”, filed Feb. 3, 2010, each of which is incorporated herein by reference in its entirety.

This invention was made with government support under Grant No. R01 GM072586 awarded by the National Institutes of Health. The government has certain rights in the invention.

### TECHNICAL FIELD

The present invention relates to triazolium catalysts useful for asymmetric synthesis, and to processes for their preparation. In particular, the invention relates to use of these compounds in asymmetric carbon-carbon bond formation. The catalysts are particularly useful when enantioselectivity is also required during the asymmetric bond formations.

### BACKGROUND OF THE INVENTION

Asymmetric carbon-carbon formation remains a formidable challenge in organic synthesis arts. Recent advances in the field of asymmetric bond formation have been limited to specific substrates with limited target substitution patterns. These limitations are most prevalent when the product of the asymmetric reaction is an enantiomeric compound. These limitations have made it particularly difficult to produce target chemical agents useful in pharmaceutical drug formation.

As such, there is a need in the field to provide safe, reactive, less hazardous, cost effective catalysts, especially catalysts capable of asymmetric carbon-carbon (C—C) bond formation.

The present invention is directed toward overcoming one or more of the problems discussed above.

### SUMMARY OF THE INVENTION

Provided herein are novel catalysts for overcoming asymmetric bond formation on a wide array of substrates. The catalysts are relatively inexpensive, versatile, and useful in providing enantioenriched products when compared to conventional methodologies.

Thus, provided herein is a novel class of bicyclic triazolium carbenc catalysts for catalytic asymmetric C—C bond formation on a variety of useful substrates. This new class of catalyst facilitates improved enantioselective control while participating in a variety of reactions with improved yield over conventional catalysts. In some embodiments, potential substrates for this new class of catalyst include both aryl and alkyl aldehydes. In other embodiments, potential substrates for this new class of catalyst include vinyl aldehydes (enals).

Also provided herein are methods for producing novel chiral triazolium catalysts (triazolium catalyst herein), and intermediates for producing the same.

Further provided herein are methods for producing a carbene form of the catalyst for use in stereocontrolled formation of carbon-carbon bonds between a variety of aldehyde and olefin substrates.

Still further provided are methods of forming C—C bond formation in a stereocontrolled manner or methods of forming asymmetric C—C bond formation. In some embodiments, the method comprises contacting an aryl aldehyde with a compound of formula (VII) and a base. In other embodiments, the method of stereocontrolled C—C bond formation comprises contacting an aryl aldehyde with a compound of formula (VII), a base, and an activated olefin. In some aspects, a nitroolefin is used as the activated olefin to form the respective 2-substituted-3-keto-arylnitropropane of formula (VIII) with unexpectedly high enantioselectivity via a Stetter reaction. In some embodiments a method of stereocontrolled C—C bond formation comprises contacting an alkyl aldehyde with a compound of formula (VII), a base, and an activated olefin. In some embodiments a trans-β-nitro-styrene is used as the activated olefin to form the respective 2-keto-arylnitroethanes of formula (VIII) in unexpectedly improved enantioselectivity via a Stetter reaction.

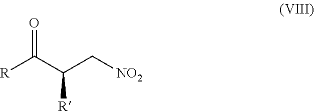

These and various other features as well as advantages, which characterize the invention, will be apparent from a reading of the following detailed description and a review of the appended claims.

### DETAILED DESCRIPTION OF THE INVENTION

N-heterocyclic carbene (NHC) catalysts have been used for carbon-carbon (C—C) bond forming reactions. NHC's with various chemical structures have been developed for further improving their performance as catalysts (see Marion, N., Díez-González, S., Nolan, S. P. Angew. Chem. Int. Ed. Engl. 2007, 46, 2988-3000; Enders, D., Niemeier, O., and Henseler, A. Chem. Rev. 2007, 107, 5606-5655; and Moore, J. and Rovis, T. Top, Curr. Chem. 2009, 290). The first chiral NHC catalysts were based on monocyclic thiazole scaffolds, formula (I), followed by bicyclic thiazolium salts, formula (II), that led to modest improvements in enantioselectivity (see Knight, R. L. and Leeper, F. J. Tetrahedron Lett. 1997, 38, 3611; Gerhard, A. U. and Leeper, F. J. Tetrahedron Lett. 1997, 38, 3615; and Dvorak, C. A. and Rawal, V. H. Tetrahedron Lett. 1998, 39, 2925). An important report described N-heterocyclic carbene (NHC) catalysts based on bicyclic triazolium salts, formula (III), by Enders in applying this to Benzoin condensation and Stetter reactions that led to further improvements in enantioselectivity (see Enders, D., Breuer, K., Runsink, J., and Teles, J. H. Helv. Chitn. Acta 1996, 79, 1899-1902; and Enders, D. and Balensiefer, T. Acc. Chem. Res. 2004, 37, 534-541). The chiral NHC's were further modified by the Rovis group in a concerted focus on structural and electronic modifications of the triazolium scaffold that imparted improved enantioselectivity and yield in both bicyclic and tetracyclic triazolium catalysts, formulas (IV) and (V), for the intramolecular Stetter reaction (see de Alaniz, J. and Rovis, T. Synlett. 2009, 1189-1207).

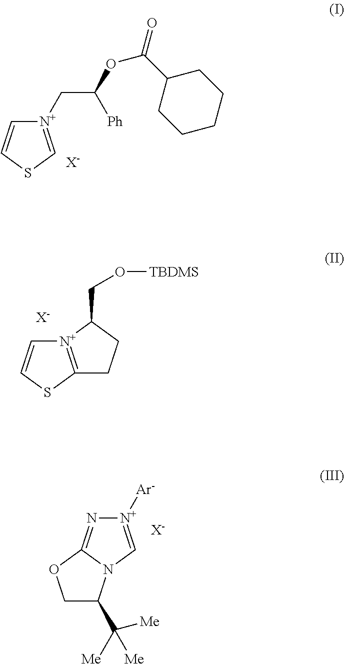

Expanding the scope of the Stetter reaction from intramolecular variants to intermolecular would allow generation of an expansive amount of substituted olefin products and thus utility. The first use of a triazolium catalyst for the intermolecular Stetter reaction achieved modest enantioselectivity of this reaction (see Enders, D., Han, J., and Henseler, A. Chem. Commun. 2008, 3989-3991; and Enders, D. and Han, J. Synthesis 2008, 3864-3868). However, the above conventional catalysts do not have sufficient catalytic activity and enantioselectivity for this reaction type or the substrate, leading to the need for improving these kinds of catalysts.

In this work the inventive approach was to create more effective catalyst species and processes that are relatively economical and safe to use compared to other known catalysts. Readily available and economical starting materials for preparation of the catalyst include substituted aryl hydrazines and pyrrolidones derived from amino acids for modification of the aryl and Z and R⁵ chiral center(s), respectively (see Kerr, M. S., Read de Alaniz, J., and Rovis, T. J. Org. Chem. 2005, 70, 5725; and Vora, H. U., Lathrop, S. P., Reynolds, N. T., Kerr, M. S., Read de Alaniz, J., and Rovis, T. Org. Synth. 2010, 87, 350). Modification of the aryl and Z and R⁵ chiral center(s) surprisingly impacted reaction efficiency (yield) and selectivity as such changes to the catalyst should have had no effect on the reaction.

Triazolium catalysts that bear a single chiral center or a second chiral center substituted with fluorine atom have been synthesized and are optically active (see DiRocco, D. A., Oberg, K. M., Dalton, D. M., and Rovis, T. J. Am. Chem. Soc. 2009, 131, 10872). Further, according to the examples in the above publication, as shown in the following Scheme I, an acyl anion, derived from the reaction of a substrate aldehyde with the carbene form of the triazolium catalyst, is subjected to an addition reaction in Michael fashion with a suitable Michael acceptor such as an activated olefin bearing an electron withdrawing group, E (so called Stetter reaction) (see Stetter, H. Angew. Chem. Int. Ed 1976, 15, 639-647).

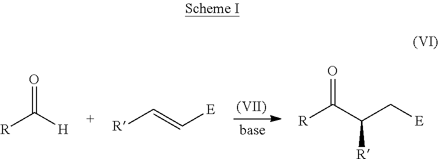

The corresponding Stetter products include 1,4-dicarbonyl compounds and related derivatives (E=keto, cyano, nitro, sulfonyl, phosphoryl, ester) such as 1,4-ketoesters, ketonitriles, and β-nitro ketones (formula VI) that are highly attractive intermediates and can be derivatized into many synthetically useful compounds due to the versatility of the functional groups (see Ono, N. The Nitro Group in Organic Synthesis; Wiley-VCH: New York, 2001).

As such, provided herein are novel asymmetric triazolium catalysts (triazolium catalyst herein), and methods for producing a carbene form that has superior characteristics as a catalyst (chemoselectivity, enantioselectivity, catalytic activity) for use in stereocontrolled formation of carbon-carbon bonds between a variety of aldehyde and olefin substrates. The resulting compounds, i.e., compounds with a chiral center adjacent to a keto group, have been shown to have tremendous potential in medical, agricultural, plastics, and other like industries.

In general, triazolium catalysts of the invention are compounds of formula (VII):

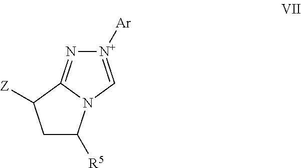

wherein Ar is an unsubstituted or substituted phenyl, naphthyl, pyridyl, pyrymidinyl, furyl, thiophenyl, pyrrolyl, or quinoline group, or any suitable heteroaromatic group. In some aspects, the Ar can be unsubstituted. In other aspects, the Ar is substituted with one or more electron-releasing or electron-withdrawing groups, for example, a substituent selected from the group consisting of X, RXₙ, RO, and NO₂, wherein R can be a substituted or unsubstituted branched or straight chain alkyl, X can be a halogen or pseudohalogen, and n is 1-3. Exemplary electron withdrawing groups include but are not limited to CH₃O, Cl, F, CF₃, NO₂ and CH₃. Z represents a halogen or pseudohalogen or electron withdrawing group and constructed in nonracemic R or S chiral isomer. Exemplary halogens, pseduohalogens, or electron withdrawing groups include but are not limited to F, Cl, Br, CN, and NO₂. R⁵ represents an H, or a substituted or unsubstituted branched or straight chain alkyl group and constructed in nonracemic R or S chiral isomer.

Also provided herein are methods for the stereocontrolled formation of C—C bonds between a variety of aldehyde and olefin substrates. One method comprises contacting an aryl aldehyde with a compound of formula (VII) and a base. In some embodiments a method of stereocontrolled C—C bond formation comprises contacting an aryl aldehyde with a compound of formula (VII) a base and an activated olefin. In some embodiments a nitroolefin is used as the activated olefin to form the respective 2-substituted-3-keto-arylnitropropane of formula (VIII) via a Stetter reaction. In some embodiments a method of stereocontrolled C—C bond formation comprises contacting an alkyl aldehyde with a compound of formula (VII) a base and an activated olefin. In some embodiments a trans-β-nitro-styrene is used as the activated olefin to form the respective 2-keto-arylnitroethanes of formula (VIII) via a Stetter reaction.

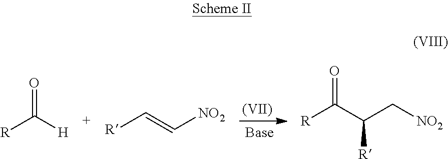

The catalysts of the invention are highly and unexpectedly versatile and capable of providing improved yields in an asymmetric manner across a wide variety of substrates. Specific fluorinated triazolium catalysts of the invention elicit an unexpected improvement in the enantioselectivity over des-fluoro counterparts. The catalysts and reaction components are relatively inexpensive compared to like reactions with conventional catalysts and methodologies. Finally, these catalysts herein are capable of turnover, thereby providing improved catalytic activity and product yield and improved enantioenriched product formation over conventional catalysts.

Still further provided are synthesis schemes for producing the catalysts described herein, as well as numerous examples that illustrate the utility of various aspects of the invention.

### The Catalysts

The catalyst species conceived of by the inventors and provided herein were generated by modification of the imidazolium rings α position(s), Z and R⁵, respectively of formula (VII). In general, use of these catalysts permit the generation of stereospecific reaction products. By increasing steric bulk of the R⁵ group relative to conventional catalysts, the new catalysts exhibit improved enantioselectivity. Triazolium catalysts with branched alkyl R⁵ groups unexpectedly achieved improved yield and enantioselectivity to as much as 90% and 88%, respectively, in the asymmetric Stetter reaction of Scheme II relative to conventional catalysts. Further inventive design was to introduce a fluorine atom Z group in conjunction with branched alkyl R⁵. This combination unexpectedly achieved catalysts with further improved yield and enantioselectivity to as much as 95% and 95%, respectively, over conventional catalysts.

Thus, provided herein are compounds of formula (VII):

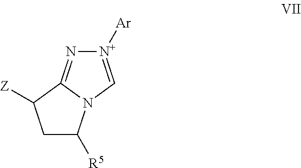

in which Ar is selected from (i) phenyl group (Ph); (ii) naphthyl; (iii) pyridyl; (iv) pyrymidinyl; (v) furyl; (vi) thiophene (vii) pyffolyl; (viii) quinoline; and (ix) any suitable heteroaromatic. Each group (i-ix) can be unsubstituted or substituted. Ar can be substituted with one or more electron-releasing or electron-withdrawing groups, for example, a substituent selected from the group consisting of X, RXₙ, RO, and NO₂, wherein R can be a substituted or unsubstituted branched or straight chain alkyl, X can be a halogen or pseudohalogen, and n is 1-3. Exemplary electron withdrawing groups include CH₃O, Cl, F, CF₃, NO₂ and CH₃.

Z is a halogen or pseudohalogen or electron withdrawing group. Chirality of Z may be R or S. Exemplary examples include F, Cl, Br, CN, and NO₂.

R⁵ can be H, or a substituted or unsubstituted branched or straight chain alkyl group. Chirality of R⁵ may be R or S.

The designations “(R)” or “R” and “(S)” or “S” are based on naming conventions well known to one of skill within the art. For example, an R-configuration is based on a compound's actual geometry, typically using the Cahn-Ingold-Prelog priority rules to classify the form (Smith M. B., March, J, March's Advanced Organic Chemistry, 5th ed. Wiley-Interscience, NY, 2001, p. 139-141).

Thus, the Ar, Z and R⁵ groups of formula (VII) can be very broad in scope. N-Alkyl substituted triazoliums including Me, n-cyclohexyl, and trifluoroethyl are also contemplated herein.

Accordingly, embodiments herein provide compounds of formulas (IX-XV):

TABLE 1
Exemplary Z and R⁵ substituted Formula (VII)

| 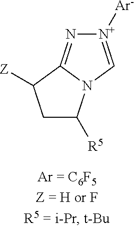 | |
|---|---|
| 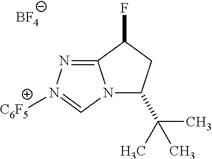 | (IX) |
| 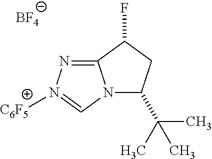 | (X) |
| 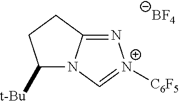 | (XII) |
| 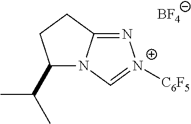 | (XI) |
| 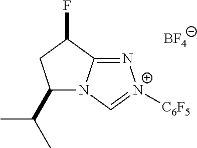 | (XIII) |
| 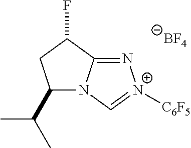 | (XIV) |
| 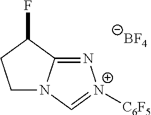 | (XV) |

TABLE 2
Illustrative Ar Groups (1-napthyl, 2-napthyl, 2-pyridyl, 3-pyridyl,
4-pyridyl, 2-pyrimidinyl, 4-pyrimidinyl, 5-pyrimidinyl,
2-furanyl, 3-furanyl, 2-thiophene, 3-thiophene):

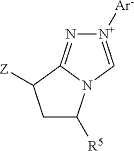

| Generic Ar Group | Potential Substituent Groups |
|---|---|
| 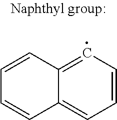 | X, RXₙ, RO and NO₂ wherein R can be a subsituted or unsubstituted branched or straight chain alkyl, X can be a halogen or pseudohalogen, and n is 1-3 |
| 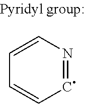 | X, RXₙ, RO, and NO₂, wherein R can be a substituted or unsubstituted branched or straight chain alkyl, X can be a halogen or pseudohalogen, and n is 1-3 |
| 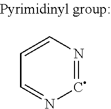 | X, RXₙ, RO, and NO₂, wherein R can be a substituted or unsubstituted branched or straight chain alkyl, X can be a halogen or pseudohalogen, and n is 1-3 |
| 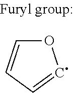 | X, RXₙ, RO, and NO₂, wherein R can be a substituted or unsubstituted branched or straight chain alkyl, X can be a halogen or pseudohalogen, and n is 1-3 |
| 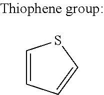 | X, RXₙ, RO, and NO₂, wherein R can be a substituted or unsubstituted branched or straight chain alkyl, X can be a halogen or pseudohalogen, and n is 1-3 |

TABLE 3
Additional Illustrative Ar Groups

| | |
|---|---|
| 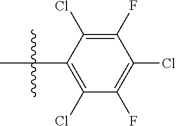 [SMILES: *c1c(Cl)c(F)c(Cl)c(F)c1Cl] | 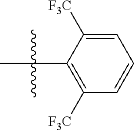 [SMILES: *c1c(C(F)(F)F)cccc1C(F)(F)F] |
| 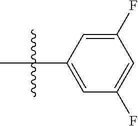 [SMILES: *c1cc(F)cc(F)c1] | 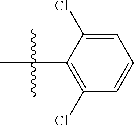 [SMILES: *c1c(Cl)cccc1Cl] |
| 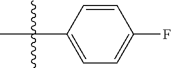 [SMILES: *c1ccc(F)cc1] | 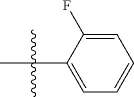 [SMILES: *c1ccccc1F] |
| 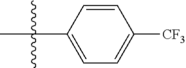 [SMILES: *c1ccc(C(F)(F)F)cc1] | 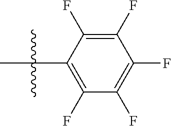 [SMILES: *c1c(F)c(F)c(F)c(F)c1F] |
| 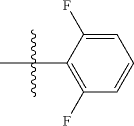 [SMILES: *c1c(F)cccc1F] | 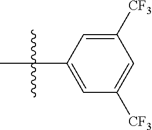 [SMILES: *c1cc(C(F)(F)F)cc(C(F)(F)F)c1] |
| 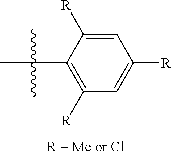 | 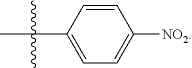 [SMILES: *c1ccc([N+](=O)[O-])cc1] |

Typical counter-ions (Y) for use with the compounds of formulas IX-XV include tetrafluoroborate (⁻BF₄), although other like charged molecules can also be used, including, for example, C₁, PF₆, BPh₄, and RBF₃.

The catalysts described herein are suitably loaded to reactions of the present invention as a mole percent of the reaction. In some embodiments the catalyst is loaded at about 1 to about 100 mole percent of the reaction, for example, about 10 to about 90 mole percent of the reaction, or about 20 to about 80 mole percent of the reaction, or about 30 to about 70 mole percent of the reaction, or about 5 to about 30 mole percent of the reaction, or about 10 to about 50 mole percent of the reaction, or about 50 to about 90 mole percent of the reaction, or about 10, about 20, about 30, or about 40 mole percent of the reaction. In other embodiments, the reaction is performed super stoichiometrically—more than 100%.

In some embodiments, a combination of catalysts can be used in a particular reaction. The combined catalysts are loaded so as to provide the same mole percent as described above, e.g., about 1 to about 100 mole percent of the reaction and so forth.

Finally, the inventive catalysts provide unexpected and surprisingly high enantioselectivity of the C—C bond formation in the generation of the products described herein. Selection of a catalyst of a particular chirality will determine the particular enantiomeric form of the product. As shown in several reactions in the examples, the chirality of the catalyst will determine the chirality of the new C—C bond formed in the Stetter reaction product. Illustratively, a catalyst of one configuration, such as that derived from (3R,5R)-3-fluoro-5-isopropylpyrrolidin-2-one, can cause addition from the bottom of the nitroolefin to produce a stereocenter of R-configuration, while a catalyst of opposite configuration, such as that derived from (3S,5S)-3-fluoro-5-isopropylpyrrolidin-2-one, can cause addition from the bottom to produce a stereocenter of S-configuration.

### Synthesis of the Catalysts

The compounds of formulas (IX)-(XV) can be prepared by methods described herein. In a first embodiment, a pyrrolidin-2-one and dichloromethane are stirred until homogeneous, then trimethyloxonium tetrafluoroborate is added in one portion and stirred for 6-18 hours at room temperature. An aryl hydrazine is then added in one portion and the mixture refluxed for 18 hours followed by solvent removal in vacuo.

In some embodiments, chlorobenzene and triethyl orthoformate or trimethyl orthoformate are added to the solution and heated to 100-130° C. in a pressure flask with stirring for 2-4 hours, with the mixture open to the atmosphere. After cooling to room temperature, this solution is then concentrated in vacuo and the resultant solid is triturated with cold ethyl acetate. The resulting off-white powder is dried under vacuum for 12 h.

In other embodiments, a catalyst can require that the hydrazide (see synthesis schematic below, third structure) be isolated before cyclization with orthoformate.

In still other embodiments, the above steps are rearranged to suit the particular synthesis reaction.

Use of other counterions can require a counterion exchange step.

The following is a general catalyst synthesis reaction (Scheme III, Y represents any suitable counterion; Ar is aromatic):

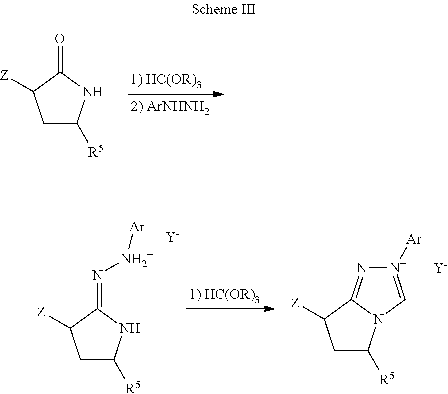

### Reactions Involving the Catalysts

Also provided herein are methods for the stereocontrolled formation of C—C bonds between a variety of aldehydes, enals, ketones, imines, unactivated alkenes, and olefin substrates in a Michael fashion (so called Stetter reaction). One embodiment comprises contacting an aryl aldehyde with a compound of formula (VII) and a base. In some aspects, a method of stereocontrolled C—C bond formation comprises contacting an aryl aldehyde with a compound of formula (VII), a base, and an activated olefin to form the compounds of formula (VIII) via a Stetter reaction. Another embodiment comprises contacting an alkyl aldehyde with a compound of formula (VII), a base, and a trans-(3-nitro-styrene to form compounds of formula (VIII) via a Stetter reaction. As described herein, any activated olefin bearing an electron withdrawing group can be used in the reaction, for example, as shown in Scheme I, above.

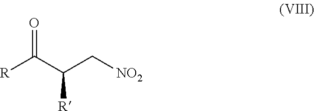

In one aspect, provided herein are methods for generating a reaction product having a specific chiral enantiomer of greater than about 50%, or greater than about 60%, or greater than about 70%, or greater than about 75%, or greater than about 80%, or greater than about 85%, or greater than about 88%, or greater than about 90%, or greater than about 91%, or greater than about 92%, or greater than about 93%, or greater than about 94%, or greater than about 95%, or greater than about 96%, or greater than about 97%, or greater than about 98%, or greater than about 99%.

Suitable aldehyde substrates include activated and unactivated aldehydes including alkyl and aryl aldehydes. Exemplary aldehydes include heteroaromatic aldehydes or alkyl aldehydes.

Suitable olefin substrates include activated olefins that have an electron withdrawing group (E) on the prochiral alkene that includes but is not limited to nitro, cyano, sulfonyl, ester, thioester, amide, keto, phosphine oxide, or phosphonate.

Each of the above asymmetric methods can be performed with a component of formula (VII) or more particularly with a compound of formula (IX)-(XV).

Also provided are methods for asymmetric formation of C—C bonds between a variety of aldehyde and olefin substrates. The method for asymmetric formation of C—C bonds comprises contacting an aldehyde, an activated olefin and a compound of formula (VII). The aldehyde can include at least one target aliphatic or aromatic functional group for formation of C—C bonds in the asymmetric reaction. The activated olefin can include at least one target aliphatic or aromatic functional group for formation of C—C bonds in the asymmetric reaction.

The reactions provided herein are amenable to a variety of substitution on the aldehyde template. R can be alkyl, cycloalkyl, aryl, and heteroaryl. In the case of alkenes, both alkene geometries may be used as well as α,β-di-substituted alkenes (R equals H). The reactions provided herein are further amenable to a variety of substitutions on the nitroolefin substrate. R′ can be substituted aryl, alkyl or cycloakyl. See also Reaction Scheme II.

The following catalysts are a representative but not exclusive subset of potential catalysts for the reaction: triazolium, thiazolium, and imidazolium catalysts with or without fused rings bearing alkyl, aryl, and heteroaryl substitution about the core as well as stereocenters in various positions.

In general, C—C bonds in the asymmetric reaction catalyzed using the compounds disclosed herein can be performed as follows: novel catalysts disclosed herein can conduct the reaction on aldehyde and olefin, in the presence of a variety of bases under polar protic solvent conditions (organic alcohol).

The reactions can occur at temperatures as low as about −40° C. or as high as about 110° C. In some embodiments, the optimal temperature for conducting the reaction is from about −10° C. to as high as ambient temperature and, in some embodiments, facilitated at about 0° C. In some embodiments, the reactions are fast and can be as short as minutes. In other embodiments, the reactions can take hours to several days to generate product.

The reaction can be performed at various scales, for example, from milligrams to grains or on a very large scale for industrial purposes or pharmaceutical manufacturing purposes.

The reaction is well suited to be conducted in polar solvents such as methanol, ethanol, isopropanol, t-amyl alcohol, and t-butanol. One can expect some degree of reaction using many different solvents, including solventless (neat), conditions.

A variety of inorganic bases or organic bases also facilitate the reaction such as but not limited to K₂CO₃, NaHCO₃, KH₂PO₄, Na₂CO₃, K₃PO₄, Et₃N, DIPEA, DBU, DBN, quinuclidine, DABCO, pyridine, Cs₂CO₃, Na₂CO₃, Li₂CO₃, NaIICO₃, KIICO₃, CsIICO₃, K₂HPO₄, KH₂PO₄, KOAc, and NaOAc.

As for equivalents relative to aldehyde and olefin substrates: catalyst from about 0.05 equivalent up to about 0.40 equivalent (for example, about 0.1 equivalent to about 0.3 equivalent, about 0.05 equivalent to about 0.2 equivalent, about 0.2 equivalent to about 0.4 equivalent, etc.), base from less than 1 equivalent to much more than one equivalent (10 or greater) (for example, about 0.5 equivalent to about 10 equivalent, about 1 equivalent to about 15 equivalent about 10 equivalent to about 18 equivalent), concentration in solvent from very dilute (0.001 M) to solventless (very concentrated) and any amount with those ranges.

When used herein, the term “halogen atom” or “halo” include fluorine, chlorine, bromine and iodine and fluoro, chloro, bromo, and iodo, respectively.

When used herein, the term “alkyl” includes all straight and branched isomers. Representative examples of these types of groups include methyl, ethyl, propyl, isopropyl, butyl, isobutyl, tert-butyl, sec-butyl, pentyl, hexyl, heptyl, and octyl.

When used herein, the term “cycloalkyl” includes cyclic isomers of the above-described alkyls. Exemplary cycloalkyls include cyclopropyl, cyclobutyl, cyclopentyl, cyclohexyl, cycloheptyl, and cyclooctyl.

“Aryl” as used herein, and unless otherwise specified, refers to an aromatic substituent containing a single aromatic ring or multiple aromatic rings that are fused together, directly linked, or indirectly linked (such that the different aromatic rings are hound to a common group such as a methylene or ethylene moiety). Exemplary aryl groups contain one aromatic ring or 2 to 4 fused or linked aromatic rings, e.g., phenyl, naphthyl, biphenyl, and the like. “Substituted aryl” refers to an aryl moiety substituted with one or more substituent groups, and the terms “heteroatom-containing aryl” and “heteroaryl” refer to aryl in which at least one carbon atom is replaced with a heteroatom. Typically the heteroaryl will contain 1-2 heteroatoms and 3-19 carbon atoms. Unless otherwise indicated, the terms “aryl” and “aromatic” includes heteroaromatic, substituted aromatic, and substituted heteroaromatic species. Illustrative aryls include phenyl, naphthyl, benxyl, tolyl, xylyl, thiophene, indolyl, etc. Illustrative heteroaryls include substituted or unsubstituted furyl, thiophenyl, pyridyl, pyrimidyl, and other heteroatom containing aromatics.

When used herein, the term “substituted” means that one or more hydrogens on the designated atom is replaced with a selection from the indicated group.

As used herein, the phrase “having the formula” or “having the structure” is not intended to be limiting and is used in the same way that the term “comprising” is commonly used.

“Stereoselective” refers to a chemical reaction that preferentially results in one stereoisomer relative to a second stereoisomer, i.e., gives rise to a product in which the ratio of a desired stereoisomer to a less desired stereoisomer is greater than 1:1. The term “stereoselective” as used herein means the same and is used interchangeably with the term “asymmetric”, for example, stereoselective C—C bond formation or asymmetric C—C bond formation.

As used herein, the phrase “enantiomeric excess” refers to the absolute difference between the mole fraction of each enantiomer.

The following is an exemplary asymmetric Stetter reaction of a heteroaromatic aldehyde and β-substituted nitroolefin followed by an exemplary asymmetric Stetter reaction of an aliphatic aldehyde and a nitrostyrene, each in accordance with embodiments described herein:

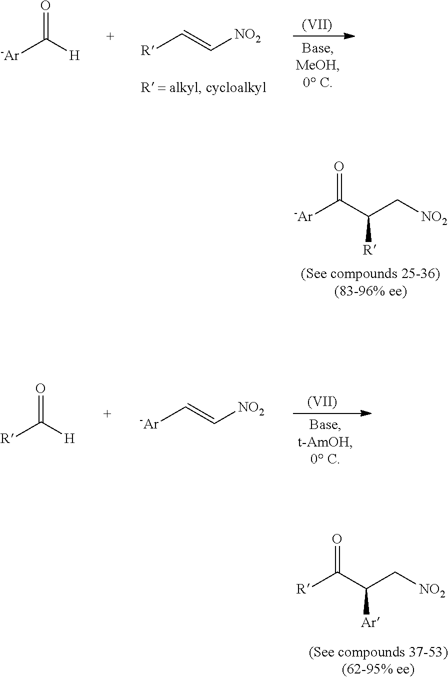

Further, the corresponding Stetter products include β-nitro ketones, examples (25-53), that are highly attractive intermediates, which can be derivatized into many synthetically useful compounds, including drug analogs, due to the versatility of the functional groups. In one embodiment a β-nitro ketone may be contacted with reducing agent such as sodium borohydride to provide the β-nitro alcohol (compound 54 below). In one embodiment a β-nitroalcohol may be contacted with a reducing agent to provide β-amino alcohol (compound 55 below) which is contacted with an acylating agent to furnish the more air stable amide (compound 56 below).

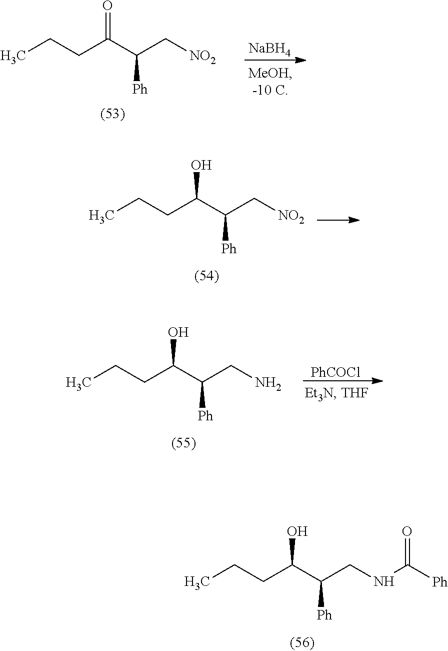 [SMILES: CCCC(=O)[C@@H](C[N+](=O)[O-])c1ccccc1 ; "PhCOCl" ; "NaBH4" ; CCC[C@@H](O)[C@@H](C[N+](=O)[O-])c1ccccc1 ; CCC[C@@H](O)[C@@H](CN)c1ccccc1 ; CCC[C@@H](O)[C@@H](CNC(=O)c1ccccc1)c1ccccc1]

Commercially important intermediates, compound (57) or (59), are available from asymmetric Stetter reactions using catalyst (XIII) via methods described herein. Compounds (57) and (59) are feasible to be converted into approved clinical agents (58) (Duloxetine) or (60) (Tramadol) via methods of nitro group reduction or keto group reduction as described herein.

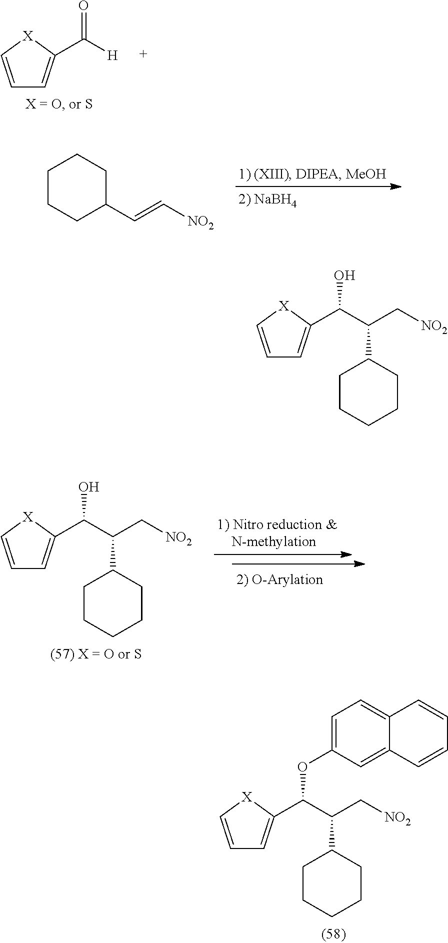

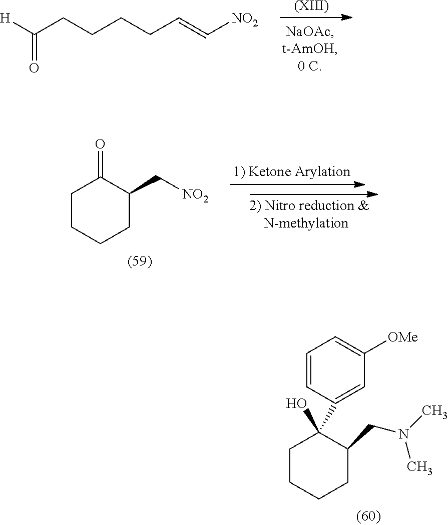

### EXAMPLES

The following examples are provided for illustrative purposes only and are not intended to limit the scope of the invention. The chart below provides abbreviations and acronyms used herein with the full word or phrase spelled out.

| | |
|---|---|
| AcOII | Acetic Acid |
| t-AmOH | tert-amyl alcohol |
| Brine | water supersaturated with sodium |
| | chloride |
| t-BuOH | Tertiary butyl alcohol |
| DCM | Dichloromethane |
| Et₃N | Trieethylamine |
| EtOAc | Ethyl acetate |
| Hex | Hexane |
| LDA | Lithium diisopropyl amide |
| Me | Methyl |
| MeOH | Methanol |
| MTBE | Methyl tertiary butyl ether |
| NCS | N-Chlorosuccinimide |
| NFSi | N-Fluorobenzenesulfonmide |
| R.T. | Room temperature |
| TBAI | Tetrabutylammonium iodide |
| THF | Tetrahydrofuran |

Synthesis reactions of exemplary Catalysts IX-XV are described below, as are the reaction products 25-53 obtained by using various exemplary catalysts and starting materials as described herein. Also shown below are the synthesis reactions of compounds 54 and 56.

### Synthesis of Triazolium Salts:

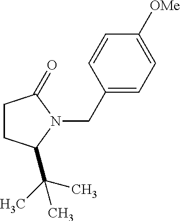 [SMILES: COc1ccc(CN2C(=O)CC[C@@H]2C(C)(C)C)cc1]

### (R)-5-(tert-butyl)-1-(4-methoxybenzyl)pyrrolidin-2-one

(16): To a dry round bottomed flask was added (R)-5-(tert-butyl)pyrrolidin-2-one (10.42 g, 73.8 mmol, 1.0 equiv) (prepared according to Smrcina, M., Majer, P., Majerova, E., Guerassina, T. and Eissenstat, M. A. Tetrahedron 1997, 53, 12867-12874) and anhydrous THF (150 mL). Sodium hydride (2.13 g, 88.6 mmol, 1.2 equiv) was added portion wise and the mixture was stirred for 30 min followed by the addition of 4-methoxybenzyl chloride (13.87 g, 88.6 mmol, 1.2 equiv) and tetrabutylammonium iodide (2.73 g, 7.38 mmol, 0.1 equiv). After 18 h a reflux condenser was installed and the reaction refluxed for 30 min. After cooling to r.t. the reaction was quenched with NH₄Cl₍ₛₐₜ₎ (100 mL), extracted with dichloromethane (3×200 mL), and dried (Na₂SO₄). Concentration of the combined organic extracts left a crude oil which was purified by flash chromatography (1:1 EtOAc:Hex) to provide the desired product (17.35 g, 90%) as a colorless oil. R_{f}=0.32 (1:1 hexanes:EtOAc); [α]_{D}²¹=80.0 (c=0.007 g/ml, CHCl₃); ¹H NMR (400 MHz, CDCl₃): δ 7.12-7.08 (m, 2H), 6.87-6.83 (m, 2H), 5.26 (d, J=15.1 Hz, 1H), 4.00 (d, J=15.1 Hz, 1H), 3.80 (s, 3H), 3.17-3.15 (m, 1H), 2.55-2.56 (m, 1H), 2.35-2.27 (m, 1H), 1.94-1.86 (m, 2H), 0.93 (s, 9H); ¹³C NMR (100 MHz, CDCl₃): δ 177.4, 159.0, 129.2, 114.1, 65.7, 55.4, 46.9, 36.9, 30.9, 27.2, 22.2; IR (NaCl, neat) 2958, 2836, 1687, 1612, 1513, 1441, 1301, 1245, 1175, 1034 cm⁻¹; HRMS (ESI+) calcd for C₁₆H₂₄NO₂ (M+H), 262.1802. Found 262.1659.

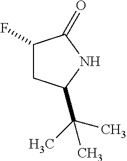

### (3S,5R)-5-(cert-butyl)-3-fluoropyrrolidin-2-one (17)

To a freshly prepared solution of LDA (1.1 equiv) in THF (300 mL) at −78° C. was added a solution of (R)-5-(tert-butyl)-1-(4-methoxybenzyl)pyrrolidin-2-one, compound (16), (17.13 g, 65.92 mmol, 1.0 equiv) in THF (100 mL) and stirred for 90 min at −78° C. A solution of NFSi (27.03 g, 85.7 mmol, 1.3 equiv) in THF (100 mL) was added dropwise, stirred for 1 h at −78° C. and then warmed to r.t. slowly by removing the dry ice/acetone bath. The reaction was quenched by the addition of NH₄Cl₍ₛₐₜ₎ (50 mL), concentrated in vacuo, and extracted with DCM (3×200 mL). The combined organic extracts were dried (Na₂SO₄) and concentrated to yield a crude solid. To this solid (mainly consisting of unreacted NFSi and benzenesulfonamide) was added ether (100 mL) while stirring vigorously. The slurry was filtered through a sintered glass funnel and washed continuously with ether (500 mL). Concentration of the filtrate in vacuo provided the crude p-methoxybenzyl(PMB)-lactam, which was immediately used in the next step without further purification. To a cooled (0° C.) solution of the crude PMB-lactam in CH₃CN (300 mL) and water (100 mL) was added eerie ammonium nitrate (90.35 g, 164.88 mmol, 2.5 equiv) portionwise. After stirring for 30 min at 0° C. the reaction was warmed to r.t., stirred an additional 1 h at r.t., and concentrated to approximately ⅓ of its original volume. Water was then added (200 mL), and the mixture extracted with DCM (3×150 mL). The combined extracts were dried (Na₂SO₄), and concentrated in vacuo to yield a crude solid. The solid was purified via flash chromatography on silica gel (20% EtOAc/hexanes) then triturated with pentanes and ether and filtered to yield the desired compound (4.85 g, 46%) as a white crystalline solid. R_{f}=0.46 (1:1 EtOAc:hex); [α]_{D}²¹=−47.0 (c=0.010 g/ml, CHCl₃); m.p. (° C.): 113-115; ¹H NMR (400 MHz, CDCl₃): δ 7.73 (bs, 1H), 5.05 (ddd, J=53.0, 7.2, 5.5 Hz, 1H), 3.53 (m, 1H), 2.27 (t, J=6.3 Hz, 1H), 2.21 (m, 1H), 0.89 (s, 9H); ¹³C NMR (100 MHz, CDCl₃): δ 173.0 (dd, J=19.9, 2.1 Hz), 88.9 (d, J=181.2 Hz), 61.6, 33.8, 30.2 (d, J=20.8 Hz), 25.5; IR (NaCl, neat) 3215, 3112, 2964, 2874, 1717, 1478, 1370, 1304, 1282, 1082 cm⁻¹; HRMS (EST+) calcd for C₈H₁₅FNO, 159.1059. Found 159.1060.

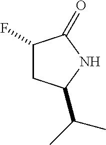

### 3S,5R)-5-(isopropyl)-3-fluoropyrrolidin-2-one (18)

prepared analogously to compound (17). ¹H-NMR (300 MHz; CDCl₃): δ 7.84 (s, 1H), 5.04 (ddd, J=52.8, 7.5, 4.5 Hz, 1H), 3.55 (q, J=5.9 Hz, 1H), 2.43-2.06 (m, 2H), 1.64 (dq, J=13.4, 6.7 Hz, 1H), 0.94 (d, J=6.7 Hz, 3H), 0.90 (d, J=6.8 Hz, 3H).

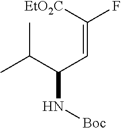 [SMILES: CCOC(=O)/C(F)=C\[C@@H](NC(=O)OC(C)(C)C)C(C)C]

### (S,E)-ethyl 4-(tert-butoxycarbonylamino)-2-fluoro-5-methylhex-2-enoate (19)

A solution of (S)-methyl 2-(tert-butoxycarbonylamino)-3-methylbutanoate (3.24 g, 14.00 mmol, 1.0 equiv) in toluene (60 mL) at −78° C. was added a 1.0 M solution of diisobutylaluminum hydride in hexanes (28.0 mL, 28.00 mmol, 2.0 equiv) dropwise. The reaction was then allowed to stir for 3 h at −78° C. at which point it was quenched with AcOH (10 mL) and then warmed slowly to room temperature. The mixture was diluted with EtOAc (100 mL) and poured into a seperatory funnel containing a 10% aqueous tartaric acid solution (100 mL). The organic layer was separated and washed with water (2×100 mL) and brine (100 mL), dried over anhydrous Na₂SO₄, and concentrated in vacuo. The crude aldehyde was dried under vacuum (4 mm) for 1 h and then used in the next step without further purification. To a solution of triethyl 2-fluoro-2-phosphonoacetate (3.56 g, 14.70 mmol, 1.05 equiv) in THF (100 mL) at room temperature was added a 1.6 M solution of n-BuLi in hexanes (9.19 mL, 14.70 mmol, 1.05 equiv) and stirred for 30 min. This solution was then cooled to −78° C. at which point a solution of the crude aldehyde (described previously) in THF (50 mL) was added dropwise via cannula over 30 min. The reaction was stirred at this temperature for 3 h and then quenched by the addition of saturated aqueous NH₄Cl (50 mL). The THF was evaporated in vacuo, and to the residue was added water (100 mL) and ELOAc (150 mL). The organic layer was washed with water (2×100 mL), dried over anhydrous Na₂SO₄, and concentrated in vacuo. The crude residue was purified by silica gel chromatography (5:1 hexanes:EtOAc) to yield the desired product as a white solid (3.34 g, 83%). R_{f}=0.43 (5:1 hexanes:EtOAc). [α]_{D}²¹=+112.4 (c=0.010 g/ml, CH₂Cl₂) m.p. (° C.): 49-50 ¹H NMR (400 MHz, CDCl₃) δ 5.73 (dd, J=21.1, 9.7 Hz, 1H), 4.83 (m, 1H), 4.63 (bs, 1H), 4.29 (m, 2H), 1.85 (bs, 1H), 1.40 (s, 9H), 0.92 (m 6H). ¹³C NMR 6 (100 MHz, CDCl₃) δ 169.1, 160.6 (J_{C-F}=35.3 Hz), 155.3, 149.0, 122.8 (m), 79.6, 61.9, 52.0, 33.1, 28.5, 19.0, 18.3, 14.3. IR (NaCl, neat) 3370, 2959, 2931, 1737, 1693, 1501, 1370 cm⁻¹. HRMS (EST+) calcd for C₁₄H₂₄FNO₄, 289.1689. Found 289.1692.

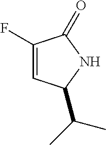

### (S)-3-fluoro-5-isopropyl-1H-pyrrol-2(5H)-one (20)

A stream of dry HCl gas was slowly bubbled through a solution of (19) (3.34 g, 11.54 mmol, 1.0 equiv) in ether (150 mL) until TLC indicated complete consumption of the starting material. The solution was then concentrated in vacuo, dissolved in toluene (150 mL), and warmed to 40° C. in a water bath. Triethylamine (4.02 mL, 28.86 mmol, 2.5 equiv) was then added dropwise over 30 min. The heterogeneous mixture was allowed to stir for an additional 30 min at which point saturated aqueous NH₄Cl (100 ml) was added. The layers were separated and the aqueous layer was then extracted with EtOAc (2×50 mL). The combined organic layers were dried over anhydrous Na₂SO₄, and concentrated in vacuo. The crude residue was then purified by silica gel chromatography (EtOAc) to yield the desired product as a white solid (1.15 g, 69%). R_{f}=0.55 (EtOAc); [α]_{D}²¹=+156.1 (c=0.010 g/ml, CH₂Cl₂) m.p. (° C.): 89-90 ¹H NMR (400 MHz. CDCl₃) δ 8.15 (bs, 1H), 6.26 (s, 1H), 3.90 (m, 1H), 1.88 (m, J=6.7 Hz, 1H), 0.95 (dd, J=5.7, 5.7 Hz, 6H); ¹³C NMR 6 (100 MHz, CDCl₃) δ 166.2 (J_{C-F}=40.0 Hz), 153.1 (J_{C-F}=278.0 Hz), 118.6 (J_{C-F}=3.5 Hz), 59.1 (J_{C-F}=5.0 Hz), 31.5, 18.5, 18.4. IR (NaCl, neat) 3201, 3125, 2954, 2927, 2868, 1698, 1655, 1462, 1194 cm⁻¹. HRMS (ESI+) calcd for C₇H₁₁FNO, 143.0747. Found 143.0746.

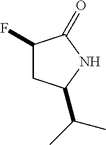

### (3R,5R)-3-fluoro-5-isopropylpyrrolidin-2-one (21)

To a solution of (20) (1.135 g, 7.93 mmol) in methanol (100 mL) was added 10% Pd/C (0.20 g) and exposed to a hydrogen atmosphere (balloon). The mixture was stirred for 12 h then filtered and concentrated in vacuo to yield the desired compound as a white solid (1.12 g, 97%). R₁=0.53 (EtOAc) [α]_{D}²¹=+92.4 (c=0.010 g/ml, CH₂Cl₂); m.p. (° C.): 92-93 ¹H NMR (400 MHz, CDCl₃) δ 7.89 (bs, 1H) 5.02 (ddd, J=52.7, 7.2, 7.2 Hz, 1H), 3.26 (m, 1H), 2.56 (m, 1H), 1.85 (m, 1H), 1.67 (m, J=6.6 Hz, 1H), 0.96 (d, J=6.6 Hz, 3H), 0.89 (d, J=6.6 Hz, 3H), ¹³C NMR 6 (100 MHz, CDCl₃) δ 173.0 (J_{C-F}=20.9 Hz), 88.8 (J_{C-F}=185.7 Hz), 32.4 (J_{C-F}=18.5 Hz), 18.9, 18.0, IR (NaCl, neat) 3217, 3104, 2975, 2863, 1709, 1698, 1473, 1328, 1296, 1081 cm⁻¹. HRMS (ESI+) calcd for C₇H₁₂FNO, 145.093. Found 145.092.

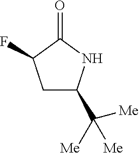 [SMILES: CC(C)(C)[C@H]1C[C@@H](F)C(=O)N1]

### (3R,5R)-5-(cert-butyl)-3-fluoropyrrolidin-2-one (22)

prepared analogously to compound (21). ¹H-NMR (300 MHz; CDCl₃): δ 7.30 (s, 1H), 5.08 (dt, J=52.8, 8.2 Hz, 1H), 3.35-3.29 (m, 1H), 2.58-2.45 (m, 1H), 1.95 (ddt, J=28.1, 13.5, 7.7 Hz, 1H), 0.93 (s, 9H).

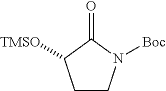

### (S)-tert-butyl 2-oxo-3-(trimethylsilyloxy)pyrrolidine-1-carboxylate (23)

To a solution of (S)-3-(trimethylsilyloxy)pyrrolidin-2-one (6.00 g, 34.62 mmol, 1.0 equiv) in CH₂Cl₂ (150 mL) was added di-tert-butyl dicarbonate (15.11 g, 69.24 mmol, 2.0 equiv), triethylamine (4.82 mL, 34.62 mmol, 1.0 equiv), and dimethylamino pyridine (4.23 g, 34.62 mmol, 1.0 equiv). The mixture was stirred overnight at room temperature then 1N HCl (100 mL) was added, and the layers were separated. The organic layer was washed with 1N HCl (2×50 mL), and brine (1×50 mL) then dried over anhydrous Na₂SO₄. The solution was concentrated in vacuo to leave a crude oil which was purified by silica gel chromatography (19:1 hexanes:EtOAc) to yield the desired compound as a clear viscous oil (7.62 g, 81%). R_{f}=0.40 (5:1, hexanes:EtOAc) [α]_{D}²¹=50.9 (c 0.010 g/ml, CH₂Cl₂); ¹H NMR (400 MHz, CDCl₃) δ 4.27 (dd, J=4.3, 4.3 Hz, 1H), 3.77 (dd, J=10.8, 9.0 Hz, 1H), 3.44 (m, 1H), 2.25 (m, 1H), 1.89 (m, 1H), 1.49 (s, 9H), 0.16 (s, 9H). ¹³C NMR 6 (100 MHz, CDCl₃) δ 172.9, 150.5, 83.2, 71.5, 42.0, 28.2, 0.3. IR (NaCl, neat) 2991, 2884, 1780, 1757, 1709, 1371, 1322, 1242, 1140 cm⁻¹. HRMS (ESI+) calcd for C₁₂H₂₃NO₄Si, 273.1396. Found 273.1393.

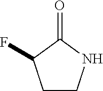

### (R)-3-fluoropyrrolidin-2-one (24)

A solution of (23) (7.62 g, 27.87 mmol, 1.0 equiv) in CH₂Cl₂ was cooled to −78° C. at which point diethylaminosulfur trifluoride (7.43 mL, 55.74 mmol, 2.0 equiv) was added dropwise. The solution was then allowed to warm to room temperature slowly and saturated aqueous NaHCO₃ (100 mL) was then added to quench the reaction. The layers were separated and the organic layer was then washed with saturated NH₄Cl (2×50 mL), dried over anhydrous Na₂SO₄, and concentrated in vacuo to yield a crude solid. This crude material was then dissolved in CH₂Cl₂ (100 mL) and trifluoroacetic acid (6.7 mL, 86.95 mmol, 3.0 equiv) was added. The solution was stirred for 3 h at which point the evolution of gas had subsided. Concentration in vacuo, then purification of the crude residue by silica gel chromatography (99:1, EtOAc:MeOH) yielded the desired product as a white solid (2.21 g, 74%). R_{f}=0.20 (EtOAc) [α]_{D}²¹=+118.7 (c=0.010 g/ml, CH₂Cl₂) m.p. (° C.): 76-78 ¹H NMR (400 MHz, CDCl₃) δ 7.70 (bs, 1H), 5.00 (ddd, J=52.7, 6.8, 6.8 Hz, 1H), 3.46 (ddd, J=9.5, 9.5, 3.6 Hz, 1H), 3.31 (m, 1H), 2.48 (m, 1H), 2.24 (m, 1H). ¹³C NMR 6 (100 MHz, CDCl₃) δ 173.5 (J_{C-F}=19.9 Hz), 88.5 (J_{C-F}=183.3 Hz), 38.9 (J_{C-F}=3.7 Hz), 28.4 (J_{C-F}=20.2 Hz). IR (NaCl, neat) 3455, 3395, 3204, 3139, 2910, 1685, 1462, 1310, 1070 cm⁻¹. HRMS (ESI+) calcd for C₄H₆FNO, 103.0433. Found 103.0439.

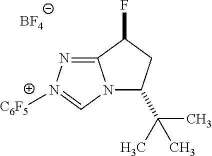

### (5S,7R)-5-(tert-butyl)-7-fluoro-2-(perfluorophenyl)-6,7-dihydro-5H-pyrrolo[2,1-c][1,2,4]triazol-2-ium tetrafluoroborate (IX)

To a flame-dried round-bottomed flask was added (3S,5R)-5-(tert-butyl)-3-fluoropyrrolidin-2-one, compound (17), (2.0 g, 12.56 mmol, 1.0 equiv) and dichloromethane (75 mL). The mixture was stirred until homogeneous then trimethyloxonium tetrafluoroborate (1.86 g, 12.56 mmol, 1.0 equiv) was added in one portion. After stirring for 18 h, pentafluorophenyl hydrazine (2.49 g, 12.56 mmol, 1.0 equiv) was added and the reaction was allowed to stir an additional 24 h. Concentration of the solution gave a solid that was triturated with ether and dried under vacuum (4 mm) for 1 h. After installing a reflux condensor, trimethyl orthoformate (20 mL) was added and the mixture was heated to reflux in an oil bath for 8 h. The solution was concentrated in vacuo and more trimethyl orthoformate (20 mL) was added. After refluxing for 18 h, this procedure was repeated once more. Finally, concentration of the solution provided gum that was crystallized with ether to yield a tan solid. The solid was filtered, washed with cold (0° C.) dichloromethane and dried to yield the desired triazolium salt (IX) (3.33 g, 60%) as a white solid. [α]_{D}²¹=+50.0 (c=0.008 g/ml, acetone); m.p. (° C.): 198-199; ¹H NMR (400 MHz, acetone): δ 10.73 (s, 1H), 6.67 (ddd, J=54.0, 7.0, 3.5 Hz, 1H), 5.32 (dd, J=7.4, 6.3 Hz, 1H), 3.46 (dddd, J=22.5, 15.3, 7.1, 6.1 Hz, 1H), 3.23 (dddd, 0.1=26.8, 15.2, 7.8, 3.6 Hz, 1H), 1.20 (s, 9H); ¹³C NMR (100 MHz, acetone): δ 161.0 (d, J=22.9 Hz), 145.0 (m), 142.5 (m), 140.1 (m), 137.4 (m), 84.2 (d, J=185.2 Hz), 72.2, 38.3 (d, J=21.9 Hz), 34.3, 25.5; IR (NaCl, neat) 3126, 2972, 2880, 1598, 1530, 1485, 1418, 1377, 1074, 1005 cm⁻¹; HRMS (ESI+) calcd for C₁₅H₁₄F₆N₃, 350.1092. Found 350.1089.

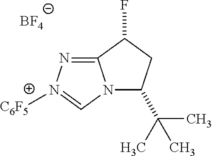

### (5S,7S)-5-(tert-butyl)-7-fluoro-2-(perfluorophenyl)-6,7-dihydro-5H-pyrrolo[2,1-c][1,2,4]triazol-2-ium tetrafluoroborate, (X)

Prepared analogously to the procedure of compound IX from (3R,5R)-5-(tert-butyl)-3-fluoropyrrolidin-2-one compound (22). White solid. [α]_{D}²¹=−3.5 (c=0.010 g/ml, acetone); m.p. (° C.): 203-204; ¹H NMR (400 MHz, acetone): δ 10.71 (s, 1H), 6.51 (ddd, J=54.8, 7.7, 1.8 Hz, 1H), 5.12 (ddd, J=8.9, 4.3, 3.4 Hz, 1H), 3.65 (dddd, J=28.0, 16.0, 8.9, 7.7 Hz, 1H), 3.07 (dddd, J=27.5, 16.0, 3.4, 1.9 Hz, 1H), 1.20 (s, 9H); ¹³C NMR (100 MHz, acetone): δ 161.0 (d, 22.7 Hz), 145.1 (m), 143.4 (m), 140.2 (m), 137.6 (m), 83.4 (d, J=184.5 Hz), 71.9, 37.5 (d, J=21.7 Hz), 34.6, 25.6; IR (NaCl, neat) 3134, 2974, 1598, 1530, 1485, 1418, 1377, 1228, 1075, 1057, 1019, 1006 cm⁻¹; HRMS (ESI+) calcd for C₁₅H₁₄F₆N₃, 350.1092. Found 350.1092.

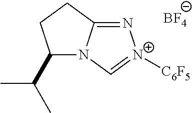

### (S)-5-isopropyl-2-(perfluorophenyl)-6,7-dihydro-5H-pyrrolo[2,1-c][1,2,4]triazol-2-ium tetrafluoroborate (XI)

To a flame-dried flask with magnetic stir bar was added (R)-5-isopropylpyrrolidin-2-one (3.28 g, 25.8 mmol) prepared according to reference (10). The flask was then evacuated and back-filled with argon. Dichloromethane (125 mL) and trimethyloxonium tetrafluoroborate (3.82 g, 25.8 mmol) were then added via powder funnel. The heterogeneous mixture was stirred at room temperature until the reaction was homogeneous (about 6 hours). Pentafluorophenyl hydrazine (5.12 g, 25.8 mmol) was added in one portion and the mixture was refluxed for 18 hours at which point dichloromethane was removed in vacuo. Triethylorthoformate (20.0 mL, 120.2 mmol) was then added and the solution transferred to a 75 mL pressure flask and heated in a 130° C. oil bath for 6 h. The resulting dark brown solution was then concentrated in vacuo to leave a semi-solid which was then triturated with ethyl acetate, filtered and washed with cold ethyl acetate. The resulting off-white powder was dried under vacuum for 12 h to give triazolium salt XI (3.21 g, 30%) as an off-white solid. [α]_{D}²¹=+30.0 (c=0.010 g/ml, MeOH); m.p. (° C.): 158-162; ¹H NMR (300 MHz, acetone-D6) δ 10.39 (s, 1H), 5.03 (m, 1H), 3.44 (m, 2H), 3.09 (m, 1H), 2.82 (m, 1H), 2.51 (m, J=6.6 Hz, 1H), 1.14 (d, J=6.8 Hz, 3H), 1.05 (d, J=6.8 Hz, 3H). ¹³C NMR (75 MHz, acetone-D6) δ 164.6, 145.1 (m), 143.8, 141.9 (m), 140.1 (m), 136.8 (m), 67.9, 31.2, 21.7, 18.0, 16.7. IR (NaCl, neat) 3125, 2979, 1600, 1527, 1061 cm⁻¹. HRMS (ESI+) calcd for C₁₄H₁₃F₅N₃, 318.1024. Found 318.1016.

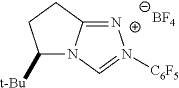

### S)-5-(tert-butyl)-2-(perfluorophenyl)-6,7-dihydro-5H-pyrrolo[2,1-c][1,2,4]triazol-2-ium tetrafluoroborate (XII)

To a flame-dried flask with magnetic stir bar was added (R)-5-tert-butylpyrrolidin-2-one (1.00 g, 7.08 mmol, 1.0 equiv), prepared according to reference (10). The flask was then evacuated and back-filled with argon. Dichloromethane (35 mL) and trimethyloxonium tetrafluoroborate (1.05 g, 7.08 mmol, 1.0 equiv) were then added via powder funnel. The heterogeneous mixture was stirred at room temperature until the reaction was homogeneous (about 6 hours). Pentafluorophenyl hydrazine (1.40 g, 7.08 mmol, 1.0 equiv) was added in one portion and the mixture was stirred for 18 hours at which point dichloromethane was removed in vacuo. Triethylorthoformate (20.0 mL) was then added and the solution transferred to a 75 mL pressure flask and heated in a 130° C. oil bath for 4 h. After cooling to room temperature, the reaction was filtered and the resultant solid was washed with ether and dried under vacuum for 12 h to give triazolium salt XII (1.60 g, 54%) as an off-white solid. [α]_{D}²¹=+28.3 (c=0.010 g/ml, MeOH); m.p. (° C.): 200-202. ¹H NMR (400 MHz, acetone-D6) δ 10.37 (s, 1H), 4.98 (dd, J=8.7, 4.8 Hz, 1H), 3.42 (m, 2H), 3.09 (m, 1H), 2.91 (m, 1H), 1.14 (s, 9H). ¹³C NMR (100 MHz, acetone-D6) δ 164.9, 144.3, 142.1 (m), 139.5 (m), 138.3 (m), 137.3 (m), 71.9, 34.6, 25.1, 21.8. IR (NaCl, neat) 3134, 2958, 2882, 1587, 1518, 1480, 1410, 1366, 1069, 1006. cm⁻¹. HRMS (ESI+) calcd for C₁₅H₁₅F₅N₃, 332.1186. Found 332.1188.

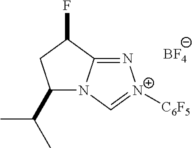

### (5S,7S)-7-fluoro-5-isopropyl-2-(perfluorophenyl)-6,7-dihydro-5H-pyrrolo[2,1-c][1,2,4]triazol-2-ium tetrafluoroborate (XIII)

To a flame-dried flask with magnetic stir bar was added compound (21) (1.00 g, 6.88 mmol, 1.0 equiv). The flask was then evacuated and back-filled with argon. Dichloromethane (50 mL) and trimethyloxonium tetrafluoroborate (1.06 g, 6.88 mmol, 1.0 equiv) were then added via powder funnel. The heterogeneous mixture was stirred at room temperature until the reaction was homogeneous (about 6 hours). Pentafluorophenyl hydrazine (1.40 g, 6.88 mmol, 1.0 equiv) was added in one portion and the mixture was refluxed for 18 hours at which point dichloromethane was removed in vacuo. Triethylorthoformate (20.0 mL) was then added and the solution transferred to a 75 mL pressure flask and heated in a 130° C. oil bath for 2 h. After cooling to room temperature, the resulting dark brown solution was then concentrated in vacuo and then chlorobenzene (40 mL) was added and the solution was heated again to 130° C. oil bath for 2 h. After cooling to room temperature, this solution was then concentrated in vacuo and the resultant solid was triturated with cold ethyl acetate. The resulting off-white powder was dried under vacuum for 12 h to give triazolium salt XIII (1.03 g, 35%) as an off-white solid. [α]_{D}²¹=+22.8 (c=0.010 g/ml, MeOH); m.p. (° C.): 154-155. ¹H NMR (400 MHz, acetone-D6) δ 10.62 (s, 1H), 6.51 (ddd, J=54.4, 7.4, 2.3 Hz, 1H), 5.12 (m, 1H), 3.61 (dddd, J=27.2, 15.7, 8.4, 7.5 Hz, 1H), 2.96 (dddd, 27.2, 15.6, 3.6, 2.3 Hz, 1H), 2.51 (m, J=6.8 Hz, 1H), 1.17 (d, J=6.8 Hz, 3H), 1.09 (d, J=6.8 Hz, 3H). ¹³C NMR (100 MHz, acetone-D6) δ 160.3 (J_{C-F}=23.1 Hz), 144.7, 144.5 (m), 141.9 (m), 139.7 (m), 137.0 (m), 83.5 (J_{C-F}=183.9 Hz), 67.3, 37.7 (J_{C-F}=21.9 Hz), 31.8, 18.0, 16.8. IR (NaCl, neat) 3136, 2965, 1704, 1607, 1543, 1478, 1065 cm⁻¹. HRMS (ESI+) calcd for C₁₄H₁₂F₆N₃, 336.0935. Found 336.0942.

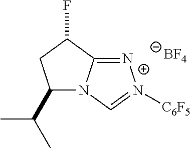

### (5S,7R)-7-fluoro-5-isopropyl-2-(perfluorophenyl)-6,7-dihydro-5H-pyrrolo[2,1-c][1,2,4]triazol-2-ium tetrafluoroborate (XIV)

Synthesized via a procedure similar to that of XIII from (3S,5R)-5-(isopropyl)-3-fluoropyrrolidin-2-one, compound (18). [α]_{D}²¹=+33.5 (c=0.010 g/ml, MeOH) m.p. (° C.): 179-181. ¹H NMR (400 MHz, acetone-D6) δ 10.65 (s, 1H), 6.61 (ddd, J=53.9, 6.7, 2.8 Hz, 1II), 5.30 (dt, 6.9, 6.8 Hz, 1II), 3.27 (m, 2H), 2.58 (m, J=6.7 Hz, 1H), 1.19 (d, J=6.8 Hz, 3H), 1.05 (d, J=6.8 Hz, 3H). ¹³C NMR (100 MHz, acetone-D6) δ 160.3 (J_{C-F}=23.4 Hz), 144.8, 144.6 (m), 139.7 (m), 137.2 (m), 83.9 (J_{C-F}=184.9 Hz), 67.5, 38.4 (J_{C-F}=22.0 Hz), 30.7, 18.1, 16.6. IR (NaCl, neat) 3142, 2975, 1709, 1591, 1527, 1478, 1071 cm⁻¹. HRMS (ESI+) calcd for C₁₄H₁₂F₆N₃, 336.0935. Found 336.0935.

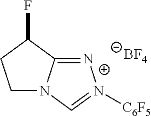

### (S)-7-fluoro-2-(perfluorophenyl)-6,7-dihydro-5H-pyrrolo[2,1-c][1,2,4]triazol-2-ium tetrafluoroborate (XV)

To a flame-dried flask with magnetic stir bar was added (24) (1.00 g, 9.70 mmol 1.0 equiv). The flask was then evacuated and back-filled with argon. Dichloromethane (50 mL) and trimethyloxonium tetrafluoroborate (1.51 g, 9.70 mmol 1.0 equiv) were then added via powder funnel. The heterogeneous mixture was stirred at room temperature until the reaction was homogeneous (about 12 hours). Pentafluorophenyl hydrazine (1.92 g, 9.70 mmol, 1.0 equiv) was added in one portion and the mixture was refluxed for 18 hours at which point dichloromethane was removed in vacuo. Chlorobenzene (40 ml) and triethylorthoformate (10.0 mL) was then added and the solution heated in a 130° C. oil bath for 12 h. The dark brown solution was then cooled to 0° C. in an ice bath and filtered. The resultant brown solid was washed with cold ethyl acetate and dried under vacuum for 12 h to give triazolium salt (XV) (2.84 g, 77%) as an off-white solid. [α]_{D}²¹=−1.8 (c=0.010 g/ml, MeOH). m.p. (° C.): 214-216. ¹H NMR (400 MHz, acetone-D6) δ 10.36 (s, 1H), 6.54 (ddd, 54.2, 7.1, 2.8 Hz, 1H), 4.97 (m, 1H), 4.87 (m, 1H), 3.50 (m, 1H), 3.11 (m, 1H). ¹³C NMR (100 MHz, acetone-D6) δ 160.8 (H_{C-F}=23.3 Hz), 144.9, 144.7 (m), 142.2 (m), 139.7 (m), 137.1 (m), 84.2 (J_{C-F}=184.8 Hz), 47.8, 35.2 (J_{C-F}=22.2 Hz). IR (NaCl, neat) 3136, 3099, 1703, 1591, 1521, 1296, 1076, 1022 cm⁻¹. HRMS (ESI+) calcd for C₁₁H₆F-₆N₃, 294.0466. Found 294.0467.

### General Procedure I for the Asymmetric Intermolecular Stetter Reaction of an Aryl Aldehyde and a Nitroolefin

To a dry 4 mL vial, with a magnetic stir bar, was added a triazolium salt of structure (VII) (0.037 mmol, 0.1 equiv). Aryl aldehyde (0.371 mmol, 1.0 equiv), β-substituted-nitroolefin (0.556 mmol, 1.5 equiv), and methanol (1 mL). The vial was then cooled to 0° C. in an ice/water bath with stirring. N,N-diisopropylethylamine (64 μl, 0.371 mmol) was added dropwise and the reaction was stirred at 0° C. for 2 h. AcOH (100 μl) was then added to quench the reaction followed by concentration in vacuo. Column chromatography (hexanes:ether) of the resulting dark red residue gave the desired β-nitro ketone.

### General Procedure II for the Asymmetric Intermolecular Stetter Reaction of Aliphatic Aldehyde and NitroStyrene:

To a dry 4 mL vial, with a magnetic stir bar, was added triazolium salt of structure (VII) (0.05 mmol, 0.2 equiv), a β-nitrostyrene (0.25 mmol, 1.0 equiv), sodium acetate (0.10 mmol, 0.4 equiv) and tert-amyl alcohol (2 ml, 0.125 M). The vial was cooled to 0° C. in a cooling bath with stirring and purged with argon. Aliphatic aldehyde (0.375 mmol, 1.5 equiv) was added dropwise and the reaction was stirred at 0° C. until TLC indicated consumption of the β-nitrostyrene (24-48 h), at which point the reaction was concentrated in vacuo. The residue was purified by flash chromatography (hexanes:ether) which provided the desired β-nitro ketone as a colorless oil.

### General Procedure III for the Synthesis of Nitroolefins

To a dry round bottom flask was added an alkyl carboxaldehyde (10.4 mmol), nitromethane (840 μl, 15.6 mmol), and 1:1 THF/t-BuOH (10 mL). This solution was cooled to 0° C. and potassium tert-butoxide (2.08 mmol) added in one portion. The reaction was then stirred at 0° C. for 1 h then warmed to room temperature and stirred for 12 h. After completion, saturated aqueous NH₄Cl solution (20 mL) was added to quench the reaction and then extracted with CH₂Cl₂ (3×20 mL). The combined organic extracts were then dried over anhydrous Na₂SO₄ and concentrated in vacuo. After drying the crude residue under vacuum (4 mm) for 1 h, CH₂Cl₂ (20 mL) was added followed by cooling to 0° C. Trifluoroacetic anhydride (10.9 mmol) was added followed by the slow dropwise addition of Et₃N (21.8 mmol). After stirring for 1 h at 0° C. the reaction was allowed to warm to room temperature and stirred an additional 2 h. The reaction was diluted with CH₂Cl₂ (20 mL) followed by the addition of water (20 mL). The organic layer was separated and washed with saturated aqueous NH₄Cl solution (3×20 mL), dried (Na₂SO₄) and concentrated in vacuo to give a yellow oil that was purified by column chromatography (20:1 hexanes:ether) yielding 0.779 g (53%) of (E)-(trans) nitroolefin as a pale yellow oil.

### Synthesis of Substituted Nitro Ketones via Asymmetric Stetter Reaction:

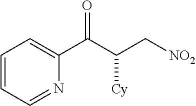

### (S)-2-cyclohexyl-3-nitro-1-(pyridin-2-yl)propan-1-one (25)

(E)-(2-nitrovinyl)cyclohexane prepared according to the general procedure III was reacted with 2-pyridinecarboxaldehyde according to general procedure I: White solid; R_{f}=0.30 (1:1 ether:hexanes) 95% yield, 95% ee; [α]_{D}²¹=−68.0 (c=0.010 g/ml, CH₂Cl₂); HPLC analysis—Chiracel OD-H column, 90:10 hexanes/iso-propanol, 1.0 mL/min. Major: 7.74 min, minor 6.90 min m.p. (° C.): 128-130 ¹H NMR (300 MHz, CDCl₃) δ 8.72 (dm, J=4.8 Hz, 1H), 8.08 (d, J=7.9 Hz, 1H), 7.86 (ddd, 0.1=7.8, 7.8, 1.8 Hz, 1H), 7.50 (ddd, J=7.8, 4.8, 1.1 Hz, 1H), 5.06 (dd, J=14.3, 10.9 Hz, 1H), 4.80 (m, 1H), 4.59 (dd, J=14.3, 3.2 Hz, 1H), 1.85 (m, 1H), 1.65 (m, 5H), 1.15 (m, 4H), 0.93 (m, 1H). ¹³C NMR (75 MHz, CDCl₃) δ 200.9, 152.7, 149.3, 137.3, 127.7, 122.7, 73.9, 47.3, 39.0, 31.5, 29.6, 26.6, 26.5, 26.2. IR (NaCl, neat) 3070, 3003, 2924, 2856, 1696, 1544, 1448, 1392 cm⁻¹. HRMS (ESI+) calcd for C₁₄H₁₉N₂O₃, 263.1390. Found 263.1393.

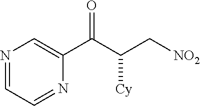

### (S)-2-cyclohexyl-3-nitro-1-(pyrazin-2-yl)propan-1-one (26)

(E)-(2-nitrovinyl)cyclohexane prepared according to the general procedure III was reacted with pyrazinecarboxaldehyde according to the general procedure I: White solid; R_{f}=0.33 (1:1 ether:hexanes); 99% yield, 96% ee; [α]_{D}²¹=−75.8 (c=0.010 g/ml, CH₂Cl₂) HPLC analysis—Chiracel OD-H column, 90:10 hexanes/iso-propanol, 1.0 mL/min. Major: 10.69 min, minor 9.67 min m.p. (° C.): 102-105 ¹H NMR (300 MHz, CDCl₃) δ 9.29 (m, 1H), 8.81 (dm, J=2.4 Hz, 1H), 8.71 (m, 1H), 5.08 (dd, J=14.6, 11.0 Hz, 1H), 4.78 (m, 1H), 4.61 (dd, J=14.6, 3.2 Hz, 1H), 1.72 (m, 6H), 1.18 (m, 4H), 0.97 (m, 1H). ¹³C NMR (75 MHz, CDCl₃) δ 200.5, 148.4, 147.0, 144.5, 143.9, 73.8, 47.4, 39.0, 31.5, 29.8, 26.6, 26.4, 26.1. IR (NaCl, neat) 3058, 2919, 2848, 1685, 1557, 1383, 1020 cm⁻¹. HRMS (ESI+) calcd for C₁₃H₁₈N₃O₃, 264.1343. Found 264.1344.

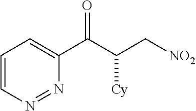

### (S)-2-cyclohexyl-3-nitro-1-(pyridazin-3-yl)propan-1-one (27)

E)-(2-nitrovinyl)cyclohexane prepared according to the general procedure III was reacted with 3-pyridazinecarboxaldehyde according to general procedure I: Yellow solid; R_{f}=0.08 (1:1 ether:hexanes); 88% yield, 94% ee; [α]_{D}²¹=−73.2. (c=0.010 g/ml, CH₂Cl₂); HPLC analysis—Chiracel OD-H column, 80:20 hexanes/iso-propanol, 1.0 mL/min. Major: 15.15 min, minor 13.10 min m.p. (° C.): 82-84; ¹H NMR (300 MHz, CDCl₃) δ 9.38 (dd, J=5.0, 1.7 Hz, 1H), 8.20 (dd, J=8.5, 1.7 Hz, 1H), 7.71 (dd, J=8.5, 5.0 Hz, 1H), 5.08 (m, 2H), 4.67 (dd, J=14.0, 2.6 Hz, 1H), 1.97 (m, 1H), 1.68 (m, 5H), 1.13 (m, 5H). ¹³C NMR (75 MHz, CDCl₃) δ 155.2, 153.6, 127.7, 125.6, 73.8, 48.0, 39.0, 31.6, 29.7, 26.5, 26.4, 26.1. IR (NaCl, neat) 2923, 2862, 1697, 1549, 1450, 1422, 1378 cm⁻¹. HRMS (ESI+) calcd for C₁₃H₁₇N₃O₃, 263.1270. Found 263.1274.

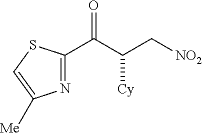

### (S)-2-cyclohexyl-1-(4-methylthiazol-2-yl)-3-nitropropan-1-one (28)

E)-(2-nitrovinyl)cyclohexane prepared according to the general procedure III was reacted with 4-Methyl-2-thiazolecarboxaldehyde according to general procedure I: White solid; R_{f}=0.65 (1:1 ether:hexanes); 70% yield, 96% ee; [α]_{D}²¹=−76.4 (c=0.010 g/ml, CH₂Cl₂) IIPLC analysis—Chiracel OD-II column, 90:10 hexanes/iso-propanol, 1.0 mL/min. Major: 7.62 min, minor: 6.97 min m.p. (° C.): 124-126; ¹H NMR (300 MHz, CDCl₃) δ 7.29 (m, 1H), 5.05 (dd, J=14.6, 11.0 Hz, 1H), 4.58 (dd, J=14.6, 3.3 Hz, 1H), 4.49 (m, 1H), 2.54 (s, 3H), 1.87 (m, 1H), 1.66 (m, 5H), 1.18 (m, 4H), 0.97 (m, 1H). ¹³C NMR 6 (75 MHz, CDCl₃) δ 193.0, 165.4, 155.9, 122.5, 73.7, 49.5, 39.2, 31.4, 29.9, 26.5, 26.4, 26.1, 17.5. IR (NaCl, neat) 3105, 2923, 2836, 1661, 1548, 1424, 1370 cm⁻¹. HRMS (ESI+) calcd for C₁₃H₁₉N₂O₃S, 283.1111. Found 283.1114.

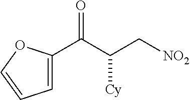

### (S)-2-cyclohexyl-1-(furan-2-yl)-3-nitropropan-1-one (29)

E)-(2-nitrovinyl)cyclohexane prepared according to the general procedure III was reacted with 2-furanylcarboxaldehyde according to general procedure 1: Clear oil; 0.28 (1:1 ether:hexanes); 75% yield, 87% ee; [α]_{D}²¹=−88.0 (c=0.010 g/ml, CH₂Cl₂); HPLC analysis—Chiracel OD-H column, 90:10 hexanes/iso-propanol, 1.0 mL/min. Major: 10.43 min, minor 8.82 min ¹H NMR (300 MHz, CDCl₃) δ 7.64 (m, 1H), 7.29 (dm, J=3.6 Hz, 1H), 6.60 (dd, J=3.6, 1.7 Hz, 1H), 5.02 (dd, J=14.6, 10.5 Hz, 1H), 4.51 (dd, J=14.6, 3.6 Hz, 1H), 3.95 (m, 1H), 1.71 (m, 6H), 1.17 (m, 4H), 0.95 (m, 1H). ¹³C NMR (75 MHz, CDCl₃) δ 188.4, 152.8, 147.2, 118.5, 112.9, 73.5, 49.9, 39.5, 31.3, 30.1, 26.5, 26.4, 26.1. IR (NaCl, neat) 3128, 2933, 2846, 1669, 1554, 1467, 1375, 1277 cm⁻¹. HRMS (ESI+) calcd for C₁₃H₁₈N₂O₄, 252.1230. Found 252.1238.

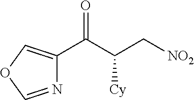

### (S)-2-cyclohexyl-3-nitro-1-(oxazol-4-yl)propan-1-one (30)

E)-(2-nitrovinyl)cyclohexane prepared according to the general procedure III was reacted with 4-oxazolecarboxaldehyde according to general procedure I: White solid; R_{f}=0.25 (1:1 ether:hexanes); 76% yield, 86% ee; [α]_{D}²¹=−83.6 (c=0.010 g/ml, CH₂Cl₂) HPLC analysis—Chiracel OD-H column, 90:10 hexanes/iso-propanol, 1.0 mL/min. Major: 12.08 min, minor 10.50 min. m.p. (° C.): 65-68; ¹H NMR (300 MHz, CDCl₃) δ 8.33 (s, 1H), 7.96 (s, 1H), 5.05 (ddd, J=14.7, 10.9, 0.7 Hz, 1H), 4.53 (ddd, J=14.7, 3.3, 0.7 Hz, 1H), 4.17 (m, 1H), 1.87 (m, 1H), 1.70 (m, 5H), 1.18 (m, 4H), 0.96 (m, 1H). ¹³C NMR (75 MHz, CDCl₃) δ 194.4, 151.2, 143.3, 140.2, 73.2, 50.5, 38.8, 31.4, 29.7, 26.5, 26.4, 26.1. IR (NaCl, neat) 2921, 2843, 1675, 1557, 1372, 1096, 1057, 905. cm⁻¹. HRMS (ESI+) calcd for C₁₂H₁₆N₂O₄, 252.1110. Found 252.1108.

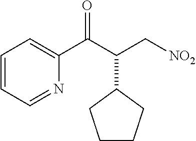 [SMILES: O=C(c1ccccn1)[C@H](C[N+](=O)[O-])C1CCCC1]

### (S)-2-cyclopentyl-3-nitro-1-(pyridin-2-yl)propan-1-one (31)

(E)-(2-nitrovinyl)cyclopentane prepared according to the general procedure III was reacted with 2-pyridinecarboxaldehyde according to general procedure I: While solid; R_{f}=0.35 (1:1 ether:hexanes); 98% yield, 90% ee; [α]_{D}²¹=−51.6 (c=0.010 g/ml, CH₂Cl₂) HPLC analysis—Chiracel OD-H column, 90:10 hexanes/iso-propanol, 1.0 mL/min. Major: 8.86 min, minor 8.11 min m.p. (° C.): 92-94; ¹H NMR (300 MHz, CDCl₃) δ 8.72 (dm, J=4.8 Hz, 1H), 8.11 (dm, J=7.8 Hz, 1H), 7.87 (ddd, J=7.7, 7.7, 1.8 Hz, 1H), 7.50 (ddd, J=7.7, 4.8, 1.2 Hz, 1H), 5.08 (dd, J=14.2, 10.7 Hz, 1H), 4.89 (ddd, J=11.8, 8.6, 3.2, 1H), 4.63 (dd, J=14.2, 3.2 Hz, 1H), 2.06 (m, 1H), 1.78 (m, 1H), 1.54 (m, 5H), 1.28 (m, 2H). ¹³C NMR (75 MHz, CDCl₃) δ 201.2, 152.9, 149.2, 137.3, 127.7, 122.8, 75.8, 46.1, 41.0, 30.7, 30.5, 25.1, 24.7. IR (NaCl, neat) 3057, 3013, 2948, 2856, 1690, 1549, 1425, 1381 cm⁻¹. HRMS (ESI+) calcd for C₁₃H₁₇N₂O₃, 249.1234. Found 249.1237.

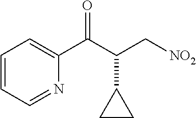 [SMILES: O=C(c1ccccn1)[C@H](C[N+](=O)[O-])C1CC1]

### (S)-2-cyclopropyl-3-nitro-1-(pyridin-2-yl)propan-1-one (32)

E)-(2-nitrovinyl)cyclopropane prepared according to the general procedure III was reacted with 2-pyridinecarboxaldehyde according to general procedure I: 72% yield, 87% ee; [α]_{D}²¹=−86.3 (c=0.006 g/ml, CH₂Cl₂) HPLC analysis—Chiracel OD-H column, 90:10 hexanes/iso-propanol, 1.0 mL/min. Major: 9.28 min, minor 8.53 min. ¹H NMR (300 MHz, CDCl₃) δ 8.72 (dm, J=4.7 Hz, 1H), 8.14 (dm, J=8.7 Hz, 1H), 7.88 (dddd, H=7.9, 7.9, 1.7, 0.4 Hz, 1H), 7.53 (dddd, J=7.6, 4.8, 1.3, 0.4 Hz, 1H), 5.15 (dd, J=14.3, 10.1 Hz, 1H), 4.72 (dd, J=14.3, 4.5 Hz, 1H), 4.25 (ddd, J=10.1, 10.1, 4.5 Hz, 1H), 0.81 (m, 1H), 0.63 (m, 2H), 0.41 (m, 2H). ¹³C NMR (75 MHz, CDCl₃) δ 199.9, 149.2, 137.3, 127.8, 123.2, 120.3, 76.2, 46.1, 11.4, 4.8, 4.4; IR (NaCl, neat) 3053, 3001, 2909, 1690, 1552, 1378, 1358 cm⁻¹. HRMS (ESI+) calcd for C₁₁H₁₃N₂O₃, 221.0921. Found 221.0923.

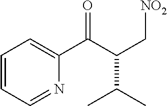 [SMILES: CC(C)[C@@H](C[N+](=O)[O-])C(=O)c1ccccn1]

### (S)-4-methyl-2-(notromethyl)-1-(pyridin-2-yl)pentan-1-one (33)

E)-(2-nitrovinyl)isopropane prepared according to the general procedure III was reacted with 2-pyridinecarboxaldehyde according to general procedure I: Prepared using 2-pyridinecarboxaldehyde and (according to the general procedure I: White solid, R_{f}=0.35 (1:1 ether:hexanes) 85% yield, 95% ee; [α]_{D}²¹=−78.0 (c=0.010 g/ml, CH₂Cl₂) HPLC analysis—Chiracel OD-H column, 90:10 hexanes/iso-propanol, 1.0 mL/min. Major: 8.52 min, minor 7.22 min; m.p. (° C.): 58-62; ¹H NMR (300 MHz, CDCl₃) δ 8.72 (m, 1H), 8.09 (ddd, J=7.9, 7.9, 0.9 Hz, 1H), 7.86 (ddd, J=7.7, 7.7, 1.7 Hz, 1H), 7.51 (dd, J=7.5, 1.1 Hz, 1H), 5.08 (dd, J=14.4, 10.8 Hz, 1H), 4.87 (ddd, J=13.9, 10.8, 5.1 Hz, 1H), 4.57 (dd, J=14.4, 3.2 Hz, 1H), 2.26 (oct, J=6.9 Hz, 1H), 1.04 (d, J=6.9 Hz, 3H), 0.88 (d, J=6.9 Hz, 3H). ¹³C NMR (75 MHz, CDCl₃) δ 200.7, 152.5, 149.3, 137.3, 127.7, 122.8, 73.3, 47.8, 29.0, 21.2, 18.8. IR (NaCl, neat) 3058, 3022, 2961, 2929, 2886, 1691, 1578, 1557, 1385 cm⁻¹. HRMS (ESI+) calcd for C₁₁H₁₅N₂O₃, 223.1077. Found 223.1073.

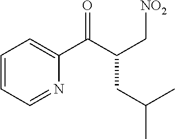 [SMILES: CC(C)C[C@@H](C[N+](=O)[O-])C(=O)c1ccccn1]

### (S)-4-methyl-2-(nitromethyl)-1-(pyridin-2-yl)pentan-1-one (34)

(E)-(2-nitrovinyl)isobutane prepared according to the general procedure III was reacted with 2-pyridinecarboxaldehyde according to general procedure I: Amorphous solid; R_{f}=0.40 (1:1 ether:hexanes); 99% yield, 83% ee; [α]_{D}²¹=−20.0 (c=0.010 g/ml, CH₂Cl₂) HPLC analysis—Chiracel OD-H column, 90:10 hexanes/iso-propanol, 1.0 mL/min. Major: 7.35 min, minor 6.93 min ¹H NMR (300 MHz, CDCl₃) δ 8.72 (d, J=4.7 Hz, 1H), 8.09 (dm, J=7.9 Hz, 1H), 7.87 (ddd, J=7.7, 7.7, 1.5 Hz, 1H), 7.51 (ddd, J=4.8, 4.8, 1.3 Hz, 1H), 4.98 (m, 2H), 4.57 (m, 1H), 1.63 (m, 2H), 1.34 (m, 1H), 0.99 (d, J=6.4 Hz, 3H), 0.93 (d, J=6.4 Hz, 3H). ¹³C NMR (75 MHz, CDCl₃) δ 201.5, 152.2, 149.3, 137.3, 127.8, 122.9, 75.8, 41.0, 38.7, 26.3, 23.1, 22.4. IR (NaCl, neat) 3059, 3020, 2952, 2924, 1690, 1583, 1544, 1380 cm⁻¹. HRMS (ESI+) calcd for C₁₂H₁₇N₂O₃, 237.1234. Found 237.1233.

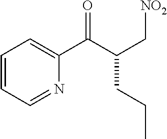 [SMILES: CCC[C@@H](C[N+](=O)[O-])C(=O)c1ccccn1]

### (S)-2-(nitromethyl)-1-(pyridin-2-yl)pentan-1-one (35)

(E)-(2-nitrovinyl)propane prepared according to the general procedure III was reacted with 2-pyridinecarboxaldehyde according to general procedure 1: Clear oil; R_{f}=0.25 (1:1 ether:hexanes); 82% yield, 83% ce; [α]_{D}²¹=+29.9. (c (0.013 g/ml, CH₂Cl₂); HPLC analysis—Chiraccl OD-H column, 90:10 hexanes/iso-propanol, 1.0 mL/min. Major: 8.30 min, minor: 7.68 min ¹H NMR (300 MHz, CDCl₃) δ 8.70 (dm, J=4.2 Hz, 1H), 8.07 (dm, J=7.9 Hz, 1H), 7.86 (ddd, J=7.7, 7.7, 1.7 Hz, 1H), 7.50 (ddd, J=7.5, 4.7, 1.2 Hz, 1H), 5.01 (dd, J=13.8, 9.7 Hz, 1H), 4.89 (m, 1H), 4.57 (dd, J=13.8, 4.0 Hz, 1H), 1.76 (m, 1H), 1.54 (m, 1H), 1.33 (m, 2H), 0.89 (t, J=7.3 Hz, 3H). ¹³C NMR (75 MHz, CDCl₃) δ 200.8, 152.2, 149.3, 137.3, 127.8, 122.9, 75.4, 42.6, 31.8, 20.3, 14.2; IR (NaCl, neat) 3054, 2952, 2930, 2868, 1696, 1549, 1386 cm⁻¹. HRMS (ESI+) calcd for C₁₁H₁₅N₂O₃, 223.1077. Found 223.1077.

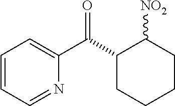

### ((1S)-2-nitrocyclohexyl)(pyridin-2-yl)methanone (36)

1-nitro-1-cyclohexene prepared according to the general procedure III was reacted with 2-pyridinecarboxaldehyde according to general procedure I: Clear oil; R_{f}=0.20 (1:1 ether:hexanes) 62% yield, 96% ee [α]_{D}²¹=−4.0, −20.3. (c=0.010 g/ml, CH₂Cl₂) HPLC analysis—Chiracel AC column, 80:20 hexanes/iso-propanol, 1.0 mL/min. Major: 13.55, 14.74 min, minor: 18.47, 16.52 min ¹H NMR (300 MHz, CDCl₃) δ 8.72 (dm. J=4.8 Hz, 1H), 8.03 (dt, J=7.8, 1.1 Hz, 1H), 7.85 (ddd, J=7.7, 7.7, 1.8 Hz, 1H), 7.49 (ddd, J=7.7, 4.8, 1.3 Hz, 1H), 4.95 (ddd, J=12.3, 10.9, 4.3 Hz, 1H), 4.61 (ddd, J=12.3, 10.9, 3.8 Hz, 1H), 2.61 (m, 1H), 2.32 (m, 1H), 1.99 (m, 1H), 1.82 (m, 2H), 1.48 (m, 2H), 1.24 (m, 1H); 6.8.63 (dm, J=4.8 Hz, 1H), 8.04 (dm, J=7.8 Hz, 1H), 7.86 (m, 1H), 7.48 (m, 1H), 5.25 (m, 1H), 4.35 (m, 1H), 2.64 (m, 1H), 2.15 (m, 1H), 1.98 (m, 2H), 1.61 (m, 4H). ¹³C NMR (75 MHz, CDCl₃) δ 201.0, 151.9, 149.3, 137.3, 127.8, 122.9, 84.9, 46.7, 31.7, 29.1, 25.1, 25.0; δ 199.6, 169.1, 152.9, 148.7, 137.5, 127.3, 123.0, 84.2, 45.4, 28.3, 24.4, 23.1, 22.3. IR (NaCl, neat) 3045, 2941, 2859, 1684, 1541, 1431, 1377 cm⁻¹, HRMS (ESI+) calcd for C₁₂H₁₅N₂O₃, 235.1077. Found 235.1077.

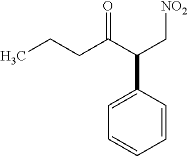

### (R)-1-nitro-2-phenylhexan-3-one (37)

Prepared using butyraldehyde and trans-β-nitrostyrene according to the general procedure II: 80% yield; 93% ee; colorless oil; R_{f}=0.24 (9:1 hex:Et₂O); [α]_{D}²¹=+299.0 (c=0.007 g/ml, CHCl₃); HPLC analysis—Chiracel IC column, 70:30 hexanes/iso-propanol, 1.0 mL/min. Major: 5.79 min, minor: 8.72 min; ¹H NMR (400 MHz, CDCl₃): δ 7.40-7.33 (m, 3H), 7.21-7.19 (m, 3H), 5.16 (dd, J=14.4, 9.2 Hz, 1H), 4.52 (dd, J=9.2, 5.2 Hz, 1H), 4.45 (dd, J=14.4, 5.2 Hz, 1H), 2.52-2.35 (m, 2H), 1.65-1.48 (m, 2H), 0.81 (t, J=7.4 Hz, 3H); ¹³C NMR (100 MHz, CDCl₃): δ 206.5, 133.1, 129.7, 128.9, 128.5, 75.4, 55.3, 43.4, 17.1, 13.6; IR (NaCl, neat) 3031, 2965, 2935, 2878, 1716, 1554, 1495, 1455, 1415, 1376, 1128, 1003 cm⁻¹; HRMS (DART) (M⁺NH₄)⁺ calcd for C₁₂H₁₉N₂O₃, 221.1052. Found 221.1058.

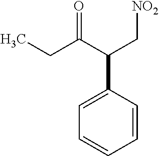

### (R)-1-nitro-2-phenylpentan-3-one (38)

Prepared using propionaldehyde and trans-β-nitrostyrene according to the general procedure II: 87% yield; 92% ee; colorless oil; R_{f}=0.16 (9:1 hex:Et₂O); [α]_{D}²¹=+379.0 (c=0.010 g/ml, CHCl₃); HPLC analysis—Chiracel IC column, 70:30 hexanes/iso-propanol, 1.0 mL/min. Major: 6.73 min, minor 9.83 min; ¹H NMR (400 MHz, CDCl₃): δ 7.40-7.32 (m, 3H), 7.21-7.19 (m, 2H), 5.16 (dd, J=14.4, 9.3 Hz, 1H), 4.54 (dd, J=9.3, 5.2 Hz, 1H), 4.46 (dd, J=14.5, 5.2 Hz, 1H), 2.57-2.41 (m, 2H), 1.02 (tᵣ=7.3 Hz, 3H); ¹³C NMR (100 MHz, CDCl₃): δ 207.1, 133.3, 129.7, 128.9, 128.5, 75.5, 55.1, 34.9, 7.8; IR (NaCl, neat) 3032, 2980, 2941, 1717, 1555, 1495, 1456, 1415, 1377, 1125, 1031 cm⁻¹; HRMS (DART) (M⁺NH₄)⁺ calcd for C₁₁H₁₇N₂O₃. 207.0895. Found 207.0900.

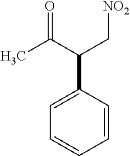

### (R)-4-nitro-3-phenylbutan-2-one (39)

Prepared using acetaldehyde and trans β-nitrostyrene according to the general procedure II: 71% yield; 62% ee; colorless oil; R_{f}=0.10 (9:1 hex:Et₂O); [α]_{D}²¹=+230.6 (c=0.012 g/ml. CHCl₃); HPLC analysis—Chiracel IC column, 70:30 hexanes/iso-propanol, 1.0 mL/min. Major: 8.49 min, minor: 9.92 min; ¹H NMR (400 MHz, CDCl₃): δ 7.42-7.34 (m, 3H), 7.22-7.20 (m, 2H), 5.14 (dd, J=14.5, 9.2 Hz, 1H), 4.54 (dd, J=9.1, 5.3 Hz, 1H), 4.45 (dd, J=14.5, 5.3 Hz, 1H), 2.17 (s, 3H); ¹³C NMR (100 MHz, CDCl₃): δ 204.2, 133.0, 129.8, 129.0, 128.5, 75.3, 56.0, 28.8; IR (NaCl, neat) 3030, 2959, 2922, 2852, 1712, 1551, 1494, 1454, 1376, 1224, 1163 cm⁻¹; HRMS (DART) (M⁺NH₄)⁺ calcd for C₁₀H₁₅N₂O₃, 193.0739. Found 193.0740.

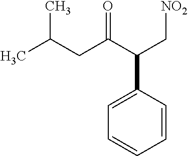

### (R)-5-methyl-1-nitro-2-phenylhexan-3-one (40)

Prepared using isovaleraldehyde and trans-p-nitrostyrene according to the general procedure II: 32% yield; 95% ee; colorless oil; R_{f}=0.27 (9:1 hex:Et₂O); [α]_{D}²¹=+276.3 (c=0.008 g/ml, CHCl₃); HPLC analysis—Chiracel IC column, 70:30 hexanes/iso-propanol, 1.0 mL/min. Major: 5.28 min, minor 8.13 min; ¹H NMR (400 MHz, CDCl₃): δ 7.40-7.33 (m, 3H), 7.19 (m, 2H), 5.15 (dd, J=14.0, 8.9 Hz, 1H), 4.51-4.42 (m, 2H), 2.40 (dd, J=16.6, 6.2 Hz, 1H), 2.25 (dd, J=16.6, 7.5 Hz, 1H), 2.13 (m, 1H), 0.88 (d, J=6.6 Hz, 3H), 0.74 (d, J=6.6 Hz, 3H); ¹³C NMR (100 MHz, CDCl₃): δ 206.0, 133.0, 129.7, 128.9, 128.6, 75.3, 55.7, 50.4, 24.3, 22.7, 22.2; IR (NaCl, neat) 3064, 3031, 2960, 2934, 2873, 1716, 1556, 1495, 1467, 1455, 1416, 1376, 1034 cm⁻¹; HRMS (DART) (M⁺NH₄)⁺ calcd for C₁₃H₂₁N₂O₃, 235.1208. Found 235.1206.

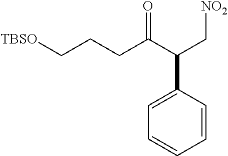

### (R)-6-((tert-butyldimethylsilyl)oxy)-1-nitro-2-phenylhexan-3-one (41)

Prepared using 4-{[t-butyl-(dimethyl)silyl]oxy)}-butyraldehyde and trans-β-nitrostyrene according to the general procedure II: 68% yield; 87% cc; colorless oil; R_{f}=0.23 (9:1 hex:Et₂O); [α]_{D}²¹=+161.5 (c=0.015 g/ml, CHCl₃); HPLC analysis—Chiracel IC column, 70:30 hexanes/iso-propanol, 1.0 mL/min. Major: 4.70 min, minor 6.01 min; ¹H NMR (400 MHz, CDCl₃): δ 7.39-7.32 (m, 3H), 7.21-7.19 (m, 2H), 5.15 (dd, J=14.4, 9.1 Hz, 1H), 4.54 (dd, J=9.1, 5.4 Hz, 1H), 4.46 (dd, J=14.4, 5.4 Hz, 1H), 3.57-3.46 (m, 2H), 2.63-2.46 (m, 3H), 1.89-1.63 (m, 3H), 0.82 (s, 9H), −0.02 (s, 3H), −0.04 (s, 3H); ¹³C NMR (100 MHz, CDCl₃): δ 206.6, 133.2, 129.8, 128.9, 128.5, 75.4, 61.8, 55.4, 37.9, 26.8, 26.0, 18.4, 5.3; IR (NaCl, neat) 2956, 2930, 2858, 1718, 1557, 1495, 1572, 1415, 1376, 1256, 1103 cm⁻¹; HRMS (DART) (M+H) calcd for C₁₈H₃₀NO₄Si, 351.1866. Found 351.1868.

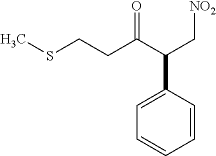

### (R)-5-(methylthio)-1-nitro-2-phenylpentan-3-one (42)

Prepared using 3-(methylthio)-propionaldehyde and trans-3-nitrostyrene according to the general procedure II: 67% yield; 92% ee; colorless oil; R_{f}=0.1 (9:1 hex:Et₂O); [α]_{D}²¹=+224.5 (c=0.011 g/ml, CHCl₃): HPLC analysis—Chiracel IC column, 70:30 hexanes/iso-propanol, 1.0 mL/min. Major: 7.96 min, minor: 10.55 min; ¹H NMR (400 MHz, CDCl₃): δ 7.41-7.34 (m, 3H), 7.22-7.20 (m, 2H), 5.16 (dd, J=14.3, 9.0 Hz, 1H), 4.56-4.45 (m, 2H), 2.83-2.60 (m, 4H), 1.99 (s, 3H); ¹³C NMR (100 MHz, CDCl₃): δ 204.7, 132.6, 129.9, 129.1, 128.6, 75.3, 55.5, 41.2, 27.8, 15.7; IR (NaCl, neat) 3030, 2964, 2920, 1715, 1552, 1494, 1414, 1375, 1111 cm⁻¹; HRMS (DART) (M⁺NH₄)⁺ calcd for C₁₂H₁₉N₂O₃S, 253.0773. Found 253.0778.

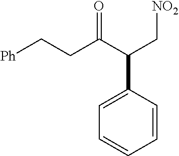

### (R)-1-nitro-2,5-diphenylpentan-3-one (43)

Prepared using 3-phenylpropanal and trans-β-nitrostyrene according to the general procedure II: 76% yield; 93% ee; colorless oil; R_{f}=0.15 (9:1 hex:Et₂O); [α]_{D}²¹=+176.8 (c=0.019 g/ml, CHCl₃); HPLC analysis—Chiracel IC column, 70:30 hexanes/iso-propanol, 1.0 mL/min. Major: 6.56 min, minor 9.60 min; ¹H NMR (400 MHz, CDCl₃): δ 7.33-7.29 (m, 3H), 7.23-7.09 (m, 5H), 7.04-7.02 (m, 2H), 5.12 (dd, J=14.0, 8.8 Hz, 1H), 4.50-4.40 (m, 2H), 2.90-2.70 (m, 4H); ¹³C NMR (100 MHz, CDCl₃): δ 205.5, 140.4, 132.8, 129.8, 128.9, 128.6, 128.5, 128.3, 126.3, 75.3, 55.5, 43.0, 29.6; IR (NaCl, neat) 3087, 3063, 3029, 2923, 1717, 1602, 1555, 1495, 1454, 1415, 1376, 1117, 1030 cm⁻¹; HRMS (DART) (M⁺NH₄)⁺ calcd for C₁₇H₂₁N₂O₃. 283.1208. Found 283.1209.

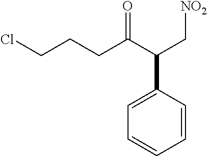

### (R)-6-chloro-1-nitro-2-phenylhexan-3-one (44)

Prepared using 4-chloro-butyraldehyde and trans-β-nitrostyrene according to the general procedure II: 83% yield; 93% ee; colorless oil; R_{f}=0.10 (9:1 hex:Et₂O); [α]_{D}²¹=+216.1 (c=0.012 g/ml, CHCl₃); HPLC analysis—Chiracel IC column, 70:30 hexanes/iso-propanol, 1.0 mL/min. Major: 6.36 min, minor 8.71 min; ¹H NMR (400 MHz, CDCl₃): δ 7.42-7.36 (m, 3H), 7.22-7.19 (m, 2H), 5.17 (dd, J=14.6, 9.5 Hz, 1H), 4.55 (dd, J=9.5, 5.0 Hz, 1H), 4.46 (dd, J=14.6, 5.0 Hz, 1H), 3.54-3.41 (m, 2H), 2.76-2.58 (m, 2H), 2.10-1.92 (m, 2H); ¹³C NMR (100 MHz. CDCl₃): δ 205.6, 132.7, 129.9, 129.1, 128.5, 75.2, 55.4, 44.1, 38.3, 26.4; IR (NaCl, neat) 3030, 2961, 2921, 1717, 1553, 1495, 1454, 1415, 1376, 1309, 1116 cm⁻¹; HRMS (DART) (M⁺NH₄)⁺ calcd for C₁₂H₁₈ClN₂O₃, 255.0662. Found 255.0665.

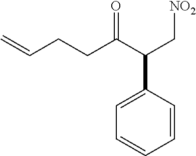

### (R)-1-nitro-2-phenylhept-6-en-3-one (45)

Prepared using 4-pentenal and trans-β-nitrostyrene according to the general procedure II: 83% yield; 93% ee; colorless oil; R_{f}=0.18 (9:1 hex:Et₂O); [α]_{D}²¹=+279.4 (c=0.007 g/ml, CHCl₃); HPLC analysis—Chiracel IC column, 70:30 hexanes/iso-propanol, 1.0 mL/min. Major: 5.81 min, minor: 8.47 min; ¹H NMR (400 MHz, CDCl₃): δ 7.41-7.33 (m, 3H), 7.19 (m, 2H), 5.73-5.63 (m, 1II), 5.16 (ddd, J=14.4, 9.3, 1.3 Hz, 1H), 4.96-4.91 (m, 2H), 4.55-4.43 (m, 2H), 2.65-2.49 (m, 2H), 2.37-2.21 (m, 2H); ¹³C NMR (100 MHz, CDCl₃): δ 205.7, 136.5, 133.0, 129.8, 129.0, 128.6, 115.6, 75.4, 55.4, 40.6, 27.5; IR (NaCl, neat) 3067, 3031, 2921, 1717, 1642, 1555, 1495, 1416, 1376, 1227, 1119 cm⁻¹; HRMS (DART) (M⁺NH₄)⁺ calcd for C_{f3}H₁₉N₂O₃, 233.1052. Found 233.1061.

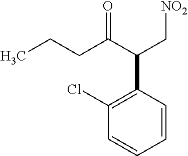

### (R)-2-(2-chlorophenyl)-1-nitrohexan-3-one (46)

Prepared using n-butyraldehyde and trans-2-chloro-P-nitrostyrene according to the general procedure II: 70% yield; 91% ee; colorless oil; R_{f}=0.22 (9:1 hex:Et₂O); [α]_{D}²¹=+232.0 (c=0.009 g/ml, CHCl₃); HPLC analysis—Chiracel IC column, 70:30 hexanes/iso-propanol, 1.0 mL/min. Major: 5.74 min, minor 7.78 min; ¹H NMR (400 MHz, CDCl₃): δ 7.46 (dd, J=7.8, 1.6 Hz, 1H), 7.30-7.22 (m, 2H), 7.07 (dd, J=7.5, 1.9 Hz, 1II), 5.14-5.04 (m, 2H), 4.43 (dd. J=13.4, 3.6 Hz, 1H), 2.47 (ddd, J=17.3, 8.0, 6.3 Hz, 1H), 2.33 (ddd, J=17.3, 7.9, 6.9 Hz, 1H), 1.64-1.49 (m, 2H), 0.81 (t, J=7.4 Hz, 3H); ¹³C NMR (100 MHz, CDCl₃): δ 205.9, 134.5, 131.1, 130.8, 130.2, 129.4, 128.0, 74.0, 51.4, 43.6, 17.1, 13.6; IR (NaCl, neat) 2966, 2934, 2878, 1719, 1556, 1475, 1416, 1376, 1130, 1052 cm⁻¹; HRMS (DART) (M⁺NH₄)⁺ calcd for C₁₂H₁₈ClN₂O₃, 255.0662. Found 255.0670.

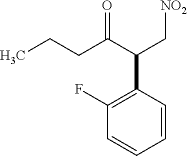 [SMILES: CCCC(=O)[C@@H](C[N+](=O)[O-])c1ccccc1F]

### (R)-2-(2-fluorophenyl)-1-nitrohexan-3-one (47)

Prepared using n-butyraldehyde and trans-2-fluoro-P-nitrostyrene according to the general procedure II: 75% yield; 93% ee; colorless oil; R_{f}=0.24 (9:1 hex:Et₂O); [α]_{D}²¹=+252.1 (c=0.011 g/ml, CHCl₃); HPLC analysis—Chiracel IC column, 70:30 hexanes/iso-propanol, 1.0 mL/min. Major: 5.50 min, minor 7.52 min; ¹H NMR (400 MHz, CDCl₃): δ 7.38-7.33 (m, 1H), 7.18-7.10 (m, 3H), 5.18 (dd, J=14.7, 9.2 Hz, 1H), 4.83 (dd, J=9.2, 5.2 Hz, 1H), 4.46 (dd, J=14.6, 5.2 Hz, 1H), 2.52-2.33 (m, 2H), 1.64-1.53 (m, 2H), 0.83 (t, J=7.4 Hz, 3H); ¹³C NMR (100 MHz, CDCl₃): δ 205.6, 160.6 (d, J=247.7 Hz), 130.8 (d, J=8.5 Hz), 129.7 (d, J=3.1 Hz), 125.3 (d, J=3.6 Hz), 120.6 (d, J=15.0 Hz), 116.6 (d. J=22.1 Hz), 74.2, 48.1, 43.2, 17.1, 13.6; IR (NaCl, neat) 2963, 2926, 1719, 1586, 1554, 1493, 1457, 1417, 1377, 1287, 1130, 1109, 1035, 1018 cm⁻¹; HRMS (DART) (M⁺NH₄)⁺ calcd for C₁₂H₁₈FN₂O₃, 239.0958. Found 239.0963

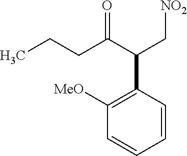 [SMILES: CCCC(=O)[C@@H](C[N+](=O)[O-])c1ccccc1OC]

### (R)-2-(2-methoxyphenyl)-1-nitrohexan-3-one (48)

Prepared using n-butyraldehyde and trans-2-methoxy-p-nitrostyrene according to the general procedure TT: 83% yield; 94% ee; colorless oil; R_{f}=0.16 (9:1 hex:Et₂O); [α]_{D}²¹=+271.5 (c=0.011 g/ml, CHCl₃); HPLC analysis—Chiracel IC column, 70:30 hexanes/iso-propanol, 1.0 mL/min. Major: 6.50 min, minor: 8.53 min; ¹H NMR (400 MHz, CDCl₃): δ 7.35-7.30 (m, 1H), 7.05 (d, J=7.6 Hz, 1H), 6.96-6.92 (m, 2H), 5.14 (dd, J=14.3, 8.7 Hz, 1H), 4.80 (dd. J=8.7, 5.4 Hz, 1H), 4.42 (dd, J=14.4, 5.4 Hz, 1H), 3.85 (s, 3H), 2.43-2.25 (m, 2H), 1.56 (m, 2H), 0.82 (t, J=7.4 Hz, 3H); ¹³C NMR (100 MHz, CDCl₃): δ 207.0, 157.0, 130.1, 129.9, 122.1, 121.4, 111.3, 74.4, 55.6, 49.9, 42.8, 17.2, 13.7; IR (NaCl, neat) 3008, 2965, 2938, 2877, 2842, 1716, 1600, 1554, 1494, 1464, 1377, 1292, 1251, 1026 cm⁻¹; HRMS (DART) (M⁺NH₄)⁺ calcd for C₁₃H₂₁N₂O₄, 251.1158. Found 251.1167.

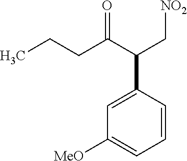 [SMILES: CCCC(=O)[C@@H](C[N+](=O)[O-])c1cccc(OC)c1]

### (R)-2-(3-methoxyphenyl)-1-nitrohexan-3-one (49)

Prepared using n-butyraldehyde and trans-3-methoxy-p-nitrostyrene according to the general procedure II: 63% yield; 91% cc; colorless oil; R_{f}=0.13 (9:1 hex:Et₂O); [α]_{D}²¹=+274.7 (c=0.006 g/ml, CHCl₃); HPLC analysis—Chiracel IC column, 70:30 hexanes/iso-propanol, 1.0 mL/min. Major: 6.56 min, minor 9.17 min; ¹H NMR (400 MHz, CDCl₃): δ 7.28 (m, 1H), 6.88 (dd, J=8.3, 2.5 Hz, 1H), 6.77 (d, J=7.6 Hz, 1H), 6.71 (t, J=2.0 Hz, 1H), 5.14 (dd, J=14.0, 8.8 Hz, 1H), 4.46 (ddd, J=18.0, 13.6, 4.8 Hz, 2H), 3.80 (s, 3H), 2.52-2.36 (m, 2H), 1.57 (m, 2H), 0.82 (t, J=7.4 Hz, 3H); ¹³C NMR (100 MHz. CDCl₃): δ 206.4, 160.5, 134.5, 130.8, 120.7, 114.3, 114.2, 75.3, 55.5, 55.3, 43.4, 17.2, 13.6; IR (NaCl, neat) 2965, 2938, 2877, 2840, 1716, 1600, 1586, 1555, 1491, 1377, 1299, 1265, 1154, 1046 cm⁻¹; HRMS (DART) (M⁺NH₄)⁺ calcd for C₁₃H₂₁N₂O₄, 251.1158. Found 251.1163.

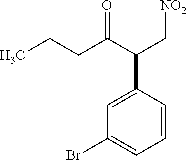 [SMILES: CCCC(=O)[C@@H](C[N+](=O)[O-])c1cccc(Br)c1]

### (R)-2-(3-bromophenyl)-1-nitrohexan-3-one (50)

Prepared using n-butyraldehyde and trans-3-bromo-β-nitrostyrene according to the general procedure II: 50% yield; 91% ee; colorless oil; R_{f}=0.17 (9:1 hex:Et₂O); 14)²¹=+244.3 (c=0.008 g/ml, CHCl₃); HPLC analysis—Chiracel IC column, 70:30 hexanes/iso-propanol, 1.0 mL/min. Major: 5.73 min, minor: 7.41 min; ¹H NMR (400 MHz, CDCl₃): δ 7.47 (ddd, J=8.0, 1.9, 1.0 Hz, 1H), 7.35 (t, J=1.8 Hz, 1H), 7.23 (t, J=7.9 Hz, 1H), 7.11 (dt, J=7.7, 1.4 Hz, 1H), 5.11 (dd, J=13.7, 8.4 Hz, 1H), 4.48-4.39 (m, 2H), 2.52-2.33 (m, 2H), 1.62-1.49 (m, 2H), 0.81 (t, J=7.4 Hz, 3H); ¹³C NMR (100 MHz, CDCl₃): δ 205.8, 135.3, 132.2, 131.6, 131.2, 127.1, 123.8, 75.2, 54.8, 43.7, 17.1, 13.6; IR (NaCl, neat) 2964, 2922, 1714, 1590, 1553, 1475, 1415, 1375, 1186, 1127, 1075, 1018 cm⁻¹; HRMS (DART) (M⁺NH₄)⁺ calcd for C₁₂H₁₈BrN₂O₃, 299.0157. Found 299.0162.

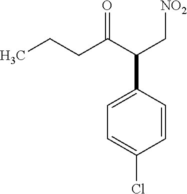 [SMILES: CCCC(=O)[C@@H](C[N+](=O)[O-])c1ccc(Cl)cc1]

### (R)-2-(4-chlorophenyl)-1-nitrohexan-3-one (51)

Prepared using n-butyraldehyde and trans-4-chloro-β-nitrostyrene according to the general procedure II: 70% yield; 92% cc; colorless oil; R_{f}=0.16 (9:1 hex:Et₂O); [α]_{D}²¹=+285.4 (c=0.010 g/ml, CHCl₃); HPLC analysis—Chiracel IC column, 70:30 hexanes/iso-propanol, 1.0 mL/min. Major: 5.58 min, minor 8.58 min; ¹H NMR (400 MHz, CDCl₃): δ 7.36 (d. J=8.5 Hz, 2H), 7.15 (d, J=8.4 Hz, 2H), 5.12 (dd, J=14.2, 8.9 Hz, 1H), 4.52-4.41 (m, 2H), 2.52-2.34 (m, 2H), 1.56 (m, 2H), 0.82 (t, J=7.4 Hz, 3H); ¹³C NMR (100 MHz, CDCl₃): δ 206.1, 135.1, 131.6, 130.0, 129.8, 75.3, 54.6, 43.6, 17.1, 13.6; IR (NaCl, neat) 2966, 2935, 2878, 1716, 1556, 1491, 1413, 1377, 1127, 1094, 1015 cm⁻¹; HRMS (DART) (M⁺NH₄)⁺ calcd for C₁₂H₁₈CIN2O₃, 255.0662. Found 255.0661.

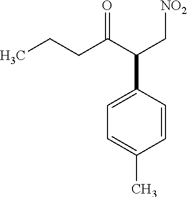 [SMILES: CCCC(=O)[C@@H](C[N+](=O)[O-])c1ccc(C)cc1]

### (R)-1-nitro-2-(p-tolyl)hexan-3-one (52)

Prepared using n-butyraldehyde and trans-4-methyl-p-nitrostyrene according to the general procedure II: 81% yield; 92% cc; colorless oil; R_{f}=0.27 (9:1 hex:Et₂O); [α]_{D}²¹=+328.4 (c=0.012 g/ml, CHCl₃); HPLC analysis—Chiracel IC column, 70:30 hexanes/iso-propanol, 1.0 mL/min. Major: 5.84 min, minor: 8.93 min; ¹H NMR (400 MHz, CDCl₃): δ 7.18 (m, 2H), 7.08 (m, 2H), 5.13 (dd, J=14.2, 9.0 Hz, 1H), 4.50-4.40 (m, 2H), 2.51-2.36 (m, 2H), 2.34 (s, 3H), 1.63-1.49 (m, 2H), 0.81 (1. J=7.4 Hz, 3H); ¹³C NMR (100 MHz, CDCl₃): δ 206.7, 138.8, 130.4, 130.1, 128.4, 75.5, 55.0, 43.3, 21.2, 17.2, 13.6; IR (NaCl, neat) 2965, 2934, 2877, 1716, 1556, 1514, 1458, 1416, 1377, 1129, 1021 cm⁻¹; HRMS (DART) (M⁺NH₄)⁺ calcd for C₁₃H₂₁N₂O₃, 235.1208. Found 235.1213.

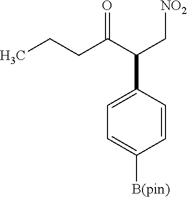

### (R)-1-nitro-2-(4-(4,4,5,5-tetramethyl-1,3,2-dioxaborolan-2-yl)phenyphexan-3-one (53)

Prepared using n-butyraldehyde and trans-4-(4-(4,4,5,5-tetramethyl-1,3,2-dioxaborolan-2-yl)phenyl)-β-nitrostyrene according to the general procedure II: 62% yield; 91% ee; colorless oil; R_{f}=0.10 (9:1 hex:Et₂O); [α]_{D}²¹=+213.3 (c=0.009 g/ml, CHCl₃); HPLC analysis—Chiracel IC column, 70:30 hexanes/iso-propanol, 1.0 mL/min. Major: 5.32 min, minor: 6.84 min; ¹H NMR (400 MHz, CDCl₃): δ 7.81 (d, J=8.1 Hz, 2H), 7.20 (d, J=8.2 Hz, 2H), 5.16 (dd, J=14.5, 9.3 Hz, 1H), 4.53 (dd, J=9.3, 5.1 Hz, 1H), 4.43 (dd, J=14.5, 5.2 Hz, 1H), 2.50-2.32 (m, 2H), 1.62-1.48 (m, 2H), 1.34 (s, 12H), 0.80 (t, J=7.4 Hz, 3H); ¹³C NMR (100 MHz, CDCl₃): δ 206.3, 136.1, 136.0, 127.9, 84.2, 75.3, 55.5, 43.5, 25.0, 17.1, 13.6; IR (NaCl, neat) 2977, 2934, 1717, 1611, 1557, 1400, 1361, 1329, 1273, 1144, 1090, 1021 cm⁻¹; HRMS (DART) (M⁺NH₄)⁺ calcd for C₁₈H₃₀BN₂O₃. 346.1940. Found 346.1944.

### Synthesis of Alcohol Products:

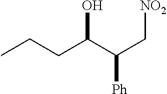 [SMILES: CCC[C@@H](O)[C@@H](C[N+](=O)[O-])c1ccccc1]

### (2R,3R)-1-nitro-2-phenylhexan-3-ol (54)

To a solution of (R)-1-nitro-2-phenylhexan-3-one (200 mg, 0.903 mmol, 1.0 equiv) in anhydrous methanol (9 mL) at −10° C. was added sodium borohydride (86 mg, 2.26 mmol, 2.5 equiv) portionwise. The reaction was stirred for 2 h at this temperature and then quenched by the addition of 10% HCl (1 mL). After stirring for 30 min the reaction was concentrated and 10% HCl (10 mL) was added. The mixture was extracted with dichloromethane (3×20 mL) and the combined organic extracts dried (Na₂SO₄) and concentrated in vacuo to yield the desired product (201 mg, 99%) in 8:1 dr. The major diastereomer was isolated by flash chromatography (15% Et₂O in hex) yielding the nitro-alcohol in >20:1 dr as a colorless oil (87%). R_{f}=0.61 (1:1 EtOAc:hex); 93% ee; [α]_{D}²¹=+17.1 (c=0.014 g/ml, CHCl₃); HPLC analysis—Chiracel IA column, 90:10 hexanes/iso-propanol, 1.0 mL/min. Major: 6.60 min, minor: 8.36 min; ¹H NMR (400 MHz, CDCl₃): δ 7.85-7.74 (m, 5H), 5.40 (dd, J=12.7, 7.4 Hz, 1H), 5.24 (dd, J=12.7, 8.0 Hz, 1H), 4.37 (m, 1H), 4.03 (td, J=7.7, 3.4 Hz, 1H), 1.98-1.89 (m, 2H), 1.89-1.77 (m, 2H), 1.71-1.61 (m, 1H), 1.39-1.33 (t, J=7.1 Hz, 3H); ¹³C NMR (100 MHz, CDCl₃): δ 135.7, 129.1, 128.9, 128.1, 77.7, 71.7, 49.2, 37.5, 19.2, 14.0; IR (NaCl, neat) 3565, 3451, 3064, 3031, 2960, 2934, 2874, 1552, 1496, 1455, 1433, 1380, 1122, 1082 cm⁻¹; HRMS (DART) (M⁺NH₄)⁺ calcd for C₁₂H₂₃N₂O₃, 223.1208. Found 223.1205.

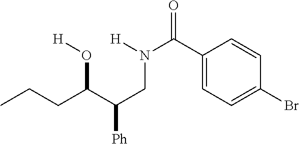 [SMILES: CCC[C@@H](O)[C@@H](CNC(=O)c1ccc(Br)cc1)c1ccccc1]

### 4-bromo-N-((2R,3R)-3-hydroxy-2-phenylhexyl)benzamide (56)

To a solution of NiCl₂-6H₂O (160 mg, 0.672 mmol, 1.5 equiv) in MeOH (5 mL) was added sodium borohydride (76 mg, 2.02 mmol, 4.5 equiv) in portions. After 30 min a solution of (2R,3R)-1-nitro-2-phenylhexan-3-ol (100 mg, 0.448 mmol, 1.0 equiv) in MeOH (1 mL) was added slowly, followed by additional sodium borohydride (60 mg, 1.56 mmol, 3.5 equiv). The heterogeneous mixture was stirred for 1 h then filtered through celite and concentrated in vacuo. The crude solid was dissolved in dichloromethane (20 mL), washed with 10% NaOH, and concentrated to yield the primary amine (55). The amine was then dissolved in THF (5 mL) and triethylamine (0.156 mL, 1.12 mmol, 2.5 equiv) was added. The solution was cooled to 0° C. at which point 4-bromobenzoyl chloride (103 mg, 0.470 mmol, 1.05 equiv) was added. After allowing the reaction to warm to room temperature, water (10 mL) and dichloromethane (10 mL) were added and the organic layer separated. The aqueous layer was extracted with dichloromethane (2×10 mL) and the combined organic extracts dried (Na₂SO₄) and concentrated to yield a solid. Trituration with ether, yielded the desired product (136 mg, 81%) as a white solid. R_{f}=0.17 (1:1 EtOAc:hex); 98% ee; [α]_{D}²¹=+17.0 (c=0.005 g/ml, acetone); HPLC analysis—Chiracel IA column, 90:10 hexanes/iso-propanol, 1.0 mL/min. Major: 12.92 min, minor 25.34 min; m.p. (° C.): 149-151; NMR (400 MHz, acetone): δ 8.11 (bs, 1H), 7.81 (m, 2H), 7.65 (m, 2H), 7.40 (m, 2H), 7.28 (m, 2H), 7.21 (m, 1H), 4.07 (dd, J=13.5, 9.6 Hz, 1H), 3.90 (m, 1H), 3.47 (dd, J=13.5, 9.6 Hz, 1H), 2.92 (ddd, J=9.5, 6.2, 3.2 Hz, 1H), 2.84 (bs, 1H), 1.44 (m, 1H), 1.28 (m, 1H), 1.16 (m, 2H), 0.78 (t, J=7.3 Hz, 3H); ¹³C NMR (100 MHz, CDCl₃): δ 167.3, 141.0, 134.4, 132.1, 130.1, 129.8, 128.5, 127.1, 125.9, 70.6, 51.8, 43.1, 37.6, 19.9, 14.1; IR (NaCl, neat) 3181, 3025, 2948, 2930, 2467, 2364, 1624, 1563, 1456, 1348, 1071 cm⁻¹; HRMS (ESI+) calcd for C₁₉H₂₃BrNO₂, 375.0834. Found 375.0830.

It is understood for purposes of this disclosure, that various changes and modifications may be made to the invention that are well within the scope of the invention. Numerous other changes may be made which will readily suggest themselves to those skilled in the art which are encompassed in the spirit of the invention disclosed herein and as defined in the appended claims

As used in the specification and the appended claims, the singular forms “a”, “an”, and “the” include plural referents unless the context clearly dictates otherwise. Thus, for example, reference to “a compound” encompasses a combination or mixture of different compounds as well as a single compound, reference to “a solvent” includes a single solvent as well as solvent mixture, and the like.

This specification contains numerous citations to references such as patents, patent applications, and publications. Each is hereby incorporated by reference for all purposes.

1. A compound of formula (VII): 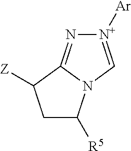
wherein Ar is an unsubstituted or substituted phenyl, naphthyl, pyridyl, pyrymidinyl, furyl, thiophenyl, quinoline, or pyrrolyl;
wherein Z is a halogen, pseudohalogen, or electron withdrawing group; and
wherein R⁵ is H, alkyl, substituted or unsubstituted branched alkyl, or substituted or unsubstituted straight chain alkyl.

2. The compound of claim 1 further comprising a counterion Y 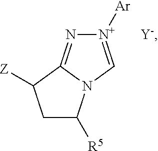
wherein the counterion is selected from the group consisting of BF₄, Cl, PF₆, BPh₄, and RBF₃.

3. The compound of claim 1, wherein the Ar is substituted phenyl.

4. The compound of claim 1, wherein Ar is phenyl group substituted with a substituent selected from the group consisting X, RXₙ, RO, and NO₂, wherein R is a substituted or unsubstituted branched or straight chain alkyl, X is a halogen or pseudohalogen, and n is 1-3.

5. The compound of claim 1, wherein the Ar is selected from the group consisting of:

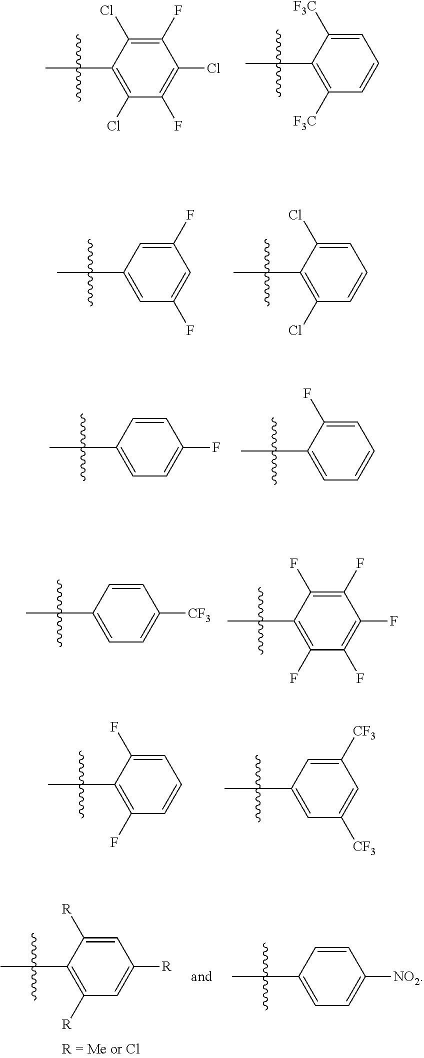

6. A composition comprising the compound of claim 1 and a base, wherein the base is selected from the group consisting of K₂CO₃, NaHCO₃, KH₂PO₄, Na₂CO₃, K₃PO₄, Et₃N, DIPEA, DBU, DBN, quinuclidine, DABCO, pyridine, Cs₂CO₃, Na₂CO₃, Li₂CO₃, NaHCO₃, KHCO₃, CsHCO₃, K₂HPO₄, KH₂PO₄, KOAc, NaOAc, and combinations thereof.

7. A method for asymmetric carbon-carbon bond formation comprising contacting an aryl aldehyde or an alkyl aldehyde with a base and a compound of formula 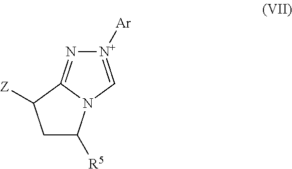
wherein Ar is an unsubstituted or substituted phenyl, naphthyl, pyridyl, pyrymidinyl, furyl, thiophenyl, quinoline, or pyrrolyl;
wherein Z is a halogen, pseudohalogen, or electron withdrawing group;
wherein R⁵ is H, alkyl, substituted or unsubstituted branched alkyl, or substituted or unsubstituted straight chain alkyl; and
wherein the asymmetric carbon-carbon bond is formed.

8. The method of claim 7, wherein Ar is phenyl group substituted with a substituent selected from the group consisting X, RXₙ, RO, and NO₂, wherein R can be a substituted or unsubstituted branched or straight chain alkyl, X can be a halogen or pseudohalogen, and n is 1-3.

9. The method of claim 7, wherein the aldehyde is an aryl aldehyde.

10. The method of claim 7, wherein the aldehyde is an alkyl aldehyde.

11. The method of claim 7, wherein the aldehyde is a heteroaromatic aldehyde or an aliphatic aldehyde.

12. The method of claim 7, further comprising contacting the aldehyde with an activated olefin having an electron withdrawing group on the prochiral alkene selected from the group consisting of nitro, cyano, sulfonyl, ester, thioester, amide, keto, phosphine oxide, and phosphonate.

13. The method of claim 7, further comprising contacting the aldehyde with an olefin, wherein the olefin is a β-substituted nitroolefin or a nitrosytrene.

14. A method for asymmetric carbon-carbon bond formation to form a β-nitro ketone, the method comprising contacting an aldehyde with a base, an olefin, and a compound of formula (VII): 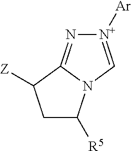
wherein Ar is an unsubstituted or substituted phenyl, naphthyl, pyridyl, pyrymidinyl, furyl, thiophenyl, quinoline, or pyrrolyl;
wherein Z is a halogen, pseudohalogen, or electron withdrawing group; and
wherein R⁵ is H, alkyl, substituted or unsubstituted branched alkyl, or substituted or unsubstituted straight chain alkyl;
wherein the respective β-nitro ketone is formed.

15. The method of claim 14, wherein Ar is phenyl group substituted with a substituent selected from the group consisting X, RXₙ, RO, and NO₂, wherein R can be a substituted or unsubstituted branched or straight chain alkyl, X can be a halogen or pseudohalogen, and n is 1-3.

16. The method of claim 14, wherein the aldehyde is an alkyl aldehyde or an aryl aldehyde.

17. The method of claim 14, wherein the base is selected from the group consisting of K₂CO₃, NaHCO₃, KH₂PO₄, Na₂CO₃, K₃PO₄, Et₃N, DIPEA, DBU, DBN, quinuclidine, DABCO, pyridine, Cs₂CO₃, Na₂CO₃, Li₂CO₃, NaHCO₃, KHCO₃, CsHCO₃, K₂HPO₄, KH₂PO₄, KOAc, NaOAc, and combinations thereof.

18. The method of claim 14, wherein the olefin is a n-substituted nitroolefin or a nitrosytrene.

19. A method for generating a (3-nitro alcohol, the method comprising contacting an aldehyde with a base, an olefin, and a compound of formula (VII): 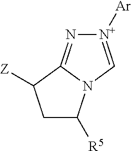
wherein Ar is an unsubstituted or substituted phenyl, naphthyl, pyridyl, pyrymidinyl, furyl, thiophenyl, quinoline, or pyrrolyl;
wherein Z is a halogen, pseudohalogen, or electron withdrawing group; and
wherein R⁵ is H, alkyl, substituted or unsubstituted branched alkyl, or substituted or unsubstituted straight chain alkyl;
to form a β-nitro ketone; and
(ii) contacting the β-nitro ketone with a reducing agent to provide the β-nitro alcohol.

20. A method for generating a n-amino alcohol, the method comprising
(i) contacting an aldehyde with a base, an olefin, and a compound of formula (VII): 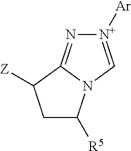
wherein Ar is an unsubstituted or substituted phenyl, naphthyl, pyridyl, pyrymidinyl, furyl, thiophenyl, quinoline, or pyrrolyl;
wherein Z is a halogen, pseudohalogen, or electron withdrawing group; and
wherein R⁵ is H, alkyl, substituted or unsubstituted branched alkyl, or substituted or unsubstituted straight chain alkyl;
to form a β-nitro ketone;
(ii) contacting the β-nitro ketone with a reducing agent to provide the β-nitro alcohol; and
(iii) contacting the β-nitro alcohol with a reducing agent to provide the n-amino alcohol.

21. The compound of claim 1, wherein the compound is (3S,5R)-5-(tert-butyl)-3-fluoropyrrolidin-2-one or (3R,5R)-5-(tert-butyl)-3-fluoropyrrolidin-2-one.

22. The method of claim 14, wherein the β-nitro ketone is selected from the group consisting of:
(R)-1-nitro-2-phenylpentan-3-one;
(R)-4-nitro-3-phenylbutan-2-one;
(R)-5-methyl-1-nitro-2-phenylhexan-3-one;
(R)-6-((tert-butyldimethylsilyl)oxy)-1-nitro-2-phenylhexan-3-one;
(R)-5-(methylthio)-1-nitro-2-phenylpentan-3-one;
(R)-1-nitro-2,5-diphenylpentan-3-one;
(R)-6-chloro-1-nitro-2-phenylhexan-3-one;
(R)-1-nitro-2-phenylhept-6-en-3-one;
(R)-2-(2-chlorophenyl)-1-nitrohexan-3-one;
(R)-2-(2-fluorophenyl)-1-nitrohexan-3-one;
(R)-2-(2-methoxyphenyl)-1-nitrohexan-3-one;
(R)-2-(3-methoxyphenyl)-1-nitrohexan-3-one;
(R)-2-(3-bromophenyl)-1-nitrohexan-3-one;
(R)-2-(4-chlorophenyl)-1-nitrohexan-3-one;
(R)-1-nitro-2-(p-tolyl)hexan-3-one; and
(R)-1-nitro-2-(4-(4,4,5,5-tetramethyl-1,3,2-dioxaborolan-2-yl)phenyl)hexan-3-one.

23. The method of claim 19, wherein the β-nitro alcohol is (2R,3R)-1-nitro-2-phenylhexan-3-ol.

24. The method of claim 20, wherein the β-amino alcohol is 4-bromo-N-((2R,3R)-3-hydroxy-2-phenylhexyl)benzamide.
